(19) United States
(12) Patent Application Publication
(10) Pub. No.: US 2007/0141141 A1
(43) Pub. Date: Jun. 21, 2007

(54) Pharmaceutical composition

(75) Inventors: Nicola Frances Bateman, Cheshire (GB); Paul Richard Gellert, Cheshire (GB); Marcel De Matas, Cheshire (GB); Alison Margaret Potts, Bedfordshire (GB)

Correspondence Address: MORGAN LEWIS & BOCKIUS LLP, 1111 PENNSYLVANIA AVENUE NW, WASHINGTON, DC, 20004, US

(73) Assignee: ASTRAZENECA AB, Sodertalje (SE)

(21) Appl. No.: 10/565,100

(22) PCT Filed: Jul. 27, 2004
(86) PCT No.: PCT/GB04/03241

§ 371 Date: Aug. 29, 2006

(30) Foreign Application Priority Data
Jul. 29, 2003 (GB) 0317663.3

(51) Int. Cl. A61K 31/5377; A61K 9/20

(52) U.S. Cl. 424/464; 514/234.2

(57) ABSTRACT

An immediate release pharmaceutical composition comprising 4-(3′-chloro-4′-fluoroanilino)-7-methoxy-6-(3-mor-pholinopropoxy)quinazoline or a pharmaceutically-acceptable salt thereof (the Agent); a water-soluble acid; and a water-soluble cellulose ether or an ester of a water-soluble cellulose ether. The claimed compositions inhibit the rate of precipitation of the Agent from solution and/or provide enhanced solubilisation of the agent at pH levels similar to those of the upper GI tract. The claimed compositions are expected to be useful in reducing inter-patient and/or intra-patient variability in exposure to the Agent.

The present invention relates to pharmaceutical compositions containing a compound with pH dependent solubility, more particularly to immediate release pharmaceutical compositions containing 4-(3′-chloro4′-fluoroanilino)-7-methoxy-6-(3-morpholinopropoxy)quinazoline or a pharmaceutically-acceptable salt thereof (hereinafter referred to as the “Agent”).

The Agent is disclosed in International Patent Application WO96/33980 (Example 1 therein) and is a potent inhibitor of the erbB family of tyrosine kinase enzymes, particularly erbB1 (EGFR, HER1). The Agent has the structure of the Formula I 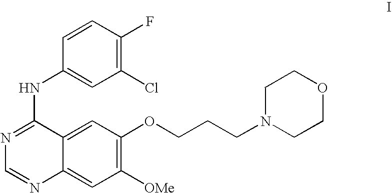
and is now known as Iressa (registered trade mark), gefitinib (Unites States Adopted Name), by way of the code number ZD1839 and Chemical Abstracts Registry Number 184475-35-2.

The Agent possesses anti-proliferative activity such as anti-cancer activity and, accordingly, is useful in methods of treatment of proliferative disease such as cancer in the human or animal body. The Agent is expected to be useful in the treatment of diseases or medical conditions mediated alone or in part by EGF (particularly erbB1) receptor tyrosine kinases, particularly cancers such as lung (especially non-small cell lung cancer), breast, prostate, ovarian, colorectal, gastric, brain, head and neck, bladder, pancreas, oesophageal, stomach, renal, skin, gynaecological and thyroid cancers and in the treatment of a range of leukaemias, lymphoid malignancies and solid tumours such as carcinomas and sarcomas. The Agent has recently been approved in a number of countries for use in the treatment of advanced non-small cell lung cancer following failure of conventional platinum based therapies.

The Agent is a weakly basic compound and has two basic groups with pKₐ's of approximately 5.3 and 7.2. The protonation and deprotonation of these basic groups has a marked effect upon the solubility of the Agent in aqueous media. Consequently, the solubility of the Agent is highly dependent upon pH. For example, the free-base form of the Agent is soluble at pH 1 (10 to 30 ml of aqueous solvent required to dissolve 1 g of Agent) but is practically insoluble above pH 7, with the solubility dropping sharply between pH 4 and pH 6 (≧10000 ml of aqueous solvent required to dissolve 1 g of Agent at pH 6).

Compounds which have pH dependent solubility, particularly basic compounds, may exhibit undesirable pharmacokinetic properties such as problems in their absorption, possibly producing low or variable bioavailability and thereby resulting inter-patient and intra-patient variability.

A factor which can affect the absorption of an orally administered drug is the changing pH experienced by the drug as it passes through the GI tract. A drug may be absorbed in a number of different sites along the GI tract following oral administration for example, the cheek lining, stomach, duodenum, jejunum, ileum and colon. The pH may be different at each site of absorption with the pH significantly different from the stomach (pH 1-3.5) to the small intestine (pH 4.5-8). When the solubility of a drug varies with pH the drug may precipitate from solution as it passes through the GI tract. This can result in variability in the extent and/or rate of absorption between doses and between patients, because the drug needs to be in solution to be absorbed.

Although the Agent has a high solubility in the acidic environment of the stomach, it is not significantly absorbed from this area. The site of highest intrinsic absorption for the Agent is thought to be the upper intestine. However, in this region of the GI tract the pH is relatively high compared to that in the stomach and the Agent has a reduced solubility at higher pH. As a result the Agent is prone to precipitate from solution as it passes from the acidic environment of the stomach to the higher pH environment of the upper GI tract (such as the upper intestine), resulting in reduced and/or variable absorption of the Agent.

The pH of the GI tract can also vary as a result of, for example, whether a patient is in a fed or fasted state, the use of certain medications, and certain medical conditions such as achlorhydria. For example, patients that use proton pump inhibitors or H2 receptor antagonists will generally have a relatively high stomach pH. These factors can result in non-uniform dissolution behaviour of a compound such as the Agent. Furthermore, the rate of gastric emptying may also influence the concentration of Agent in solution that reaches the principle absorption sites in the GI tract.

For example a high rate of gastric emptying or a locally high pH in the stomach when a pharmaceutical composition containing the Agent is administered to a patient may result in only partial dissolution of the Agent in the stomach. The remaining un-dissolved portion of the composition will then be transferred to the higher pH environment of the upper intestine where further dissolution of the Agent is inhibited because of the low solubility of the Agent at higher pH. Accordingly, the combination of the sensitive pH solubility profile of the Agent together with the variability of the pH in the GI tract and/or rate of gastric emptying may result in a high degree of inter-patient variability in the bioavailability and/or plasma concentrations of the Agent and possibly sub-optimal treatment efficacy in a proportion of patients. There is therefore a need to reduce the pH sensitivity of the Agent and thereby provide a more uniform delivery of high concentrations of the Agent to the major absorption sites for the Agent in the GI tract.

U.S. Pat. No. 4,344,934 describes a pharmaceutical composition comprising wetted mixtures of a poorly water-soluble drug and a water-soluble polymer which are stated to exhibit improved bio-availability.

GB 2,306,885 describes a topical composition containing a drug with pH dependent solubility, wherein the composition becomes supersaturated with the drug when the composition is applied to the skin as a result of the change in pH. The compositions optionally contain an anti-nucleating agent to inhibit precipitation of the drug from the composition.

Usui et al (Int. J. Pharmaceutics 154 (1997) 59-66) found that certain water-soluble polymers inhibited the precipitation of a specific compound, RS-8359, from supersaturated aqueous methanol solutions.

Loftsson et al (Int. J. Pharmaceutics 127 (1996) 293-296) describe the effects of water-soluble polymers on drug solubility of the compounds acetazolamide, hydrocortisone, prazepam and sulfamethoxazole.

WO 95/09614 describes a composition comprising an HIV protease inhibitor dissolved/mixed with an organic solvent and an organic acid and the resultant mixture is adsorbed onto a suitable adsorbent substrate. The compositions are stated to exhibit enhanced bioavailability.

Venkatesh (Pharmaceutical Development & Technology, 3(4), 477485 (1998)), WO 02/06834 and WO 03/24429 all describe the use of acids to control solubility and release of drugs from controlled/sustained release formulations.

U.S. Pat. No. 6,248,771 discloses a composition containing a hydrophobic quinazoline compound a “polyoxyhydrocarbyl compound” such as a polyethylene glycol derivative and a surfactant. WO 00/59475 discloses a composition comprising a hydrophobic therapeutic agent having at least one ionizable group dissolved in a carrier comprising an ionizing agent and a surfactant. The compositions described contain high levels of surfactant which acts to retain the therapeutic agent in solution in-vivo. However, compositions containing high levels of surfactants are difficult to formulate. Furthermore the administration of high levels of surfactant to patients can result in GI irritation and/or disturbances.

EP 640 341 discloses a controlled release composition comprising a core containing a specific pyridinedione compound, an organic acid and a water-soluble polymer; and an enteric coating. The composition is designed to be insoluble in the stomach and to dissolve in the intestine. The organic acid is stated to act to rapidly dissolve the pyridinedione in the alkaline/neutral environment of the intestine whilst the polymer retains the compound in solution and the enteric coating controls release of the polymer/pyridinedione compound over a period of typically 24 hours.

WO 01/47495 discloses a composition comprising a drug in a solubility enhanced form and a concentration enhancing polymer. The solubility-enhanced form of the drug includes soluble salts, polymorphs or solubilised forms of a drug. The initial high concentration obtained upon dissolution of the solubility-enhanced form of the drug is maintained by means of the concentration enhancing polymer. Generally the compositions provide an increase in maximum concentration (Cₘₐₓ) and/or area under the curve (AUC) of the order 1.25 or more compared to the drug in a low solubility form.

Co-pending PCT application no PCT/GB03/00803, published as WO 03/072139 after the priority date of the present application discloses compositions comprising the Agent and a water-soluble cellulose ether or an ester of a water-soluble cellulose ether. The compositions described therein inhibit the rate of precipitation of the Agent from solution following an increase in pH and thereby provide an increased concentration of Agent in solution at higher pH compared to compositions that do not contain a water-soluble cellulose ether or an ester of a water-soluble cellulose ether.

There is a need for improved compositions containing the Agent, particularly compositions which provide high levels of the Agent in solution at the site of absorption of the Agent.

We have surprisingly found that compositions comprising the Agent, certain polymers and an acid provide high concentrations of the agent at pH levels similar to those found in the upper GI tract (such as the upper intestine). The compositions according to the present invention provide substantially pH independent delivery of the Agent and an increased concentration of the Agent at the major absorption site. The increased concentration of Agent is expected to provide improved pharmacokinetic properties for example, increased extent of absorption and/or bioavailability and may reduce inter-patient variability in the bioavailability and/or variable plasma concentrations of the Agent.

According to a first aspect of the present invention there is provided an immediate release pharmaceutical composition comprising the Agent, a water-soluble acid and a water-soluble cellulose ether or an ester of a water-soluble cellulose ether.

According to another aspect of the present invention there is provided an immediate release pharmaceutical composition comprising the Agent, a water-soluble acid and a water-soluble cellulose ether.

According to another aspect of the present invention there is provided an immediate release pharmaceutical composition comprising the Agent, a water-soluble organic acid and a water-soluble cellulose ether.

According to another aspect of the present invention there is provided an immediate release pharmaceutical composition comprising the Agent, a water-soluble acid and an ester of a water-soluble cellulose ether.

According to another aspect of the present invention there is provided an immediate release pharmaceutical composition comprising the Agent, a water-soluble organic acid and an ester of a water-soluble cellulose ether.

We have found that administration of the composition to a low pH environment such as the stomach results in the rapid dissolution of the Agent as expected. However, upon an increase in pH (for example as occurs when the Agent passes from the stomach to the upper intestine), surprisingly high levels of the Agent remain in solution, despite the highly sensitive pH solubility profile of the Agent.

We have also found that the compositions according to the invention provide surprisingly enhanced solubilisation of the Agent at pH values similar to those found in the upper GI tract (for example pH 4.5 to 8) compared to the solubilisation of any of: (i) the Agent alone; (ii) solubilisation of the Agent from a composition containing the Agent and the water-soluble cellulose ether or ester thereof; or (iii) solubilisation of the Agent from a composition containing the Agent and the water-soluble acid.

The dissolution properties of the composition according to the invention are such that in the event that any of the composition does not dissolve in the stomach (for example as a result of rapid gastric emptying) the improved solubilisation at higher pH will increase the concentration of the Agent at the higher pH environment of the upper intestine. Accordingly, the present invention provides a means for essentially pH independent delivery of high concentrations of the Agent to the major absorption sites of the Agent in the GI tract. An essentially pH independent delivery of the Agent is expected to reduce the variability in bioavailability between patients, thereby enabling the dose of Agent required to give a therapeutic effect to be minimised. This in turn may reduce undesirable side effects which may be associated with high doses of the Agent.

By the term “enhanced solubilisation” of the Agent is meant that when a composition according to the invention is placed in a aqueous medium the dissolution of the Agent from the composition of the invention is increased compared to dissolution of the Agent alone. For example, the compositions according to the invention give at least a 3 fold increase in the maximum concentration (Cmax) following dissolution at pH 6.5 compared to the equilibrium concentration of the Agent alone without the water-soluble cellulose ether or acid. In preferred embodiments the composition provides at least a 5 fold, more preferably at least a 7 fold and still more preferably at least a 10 fold increase in Cmax compared to the equilibrium concentration of the Agent alone. For the purposes of the present invention, the equilibrium concentration of the Agent alone may be approximated to the concentration of the Agent in solution 60 minutes following introduction of the Agent to a dissolution medium at pH 6.5 and 37° C.

In embodiments of the invention the composition of the invention provides an area under the dissolution versus time curve (AUC) of at least 3 times, preferably at least 5 times, more preferably at least 10 and especially 10 to 50 times the AUC of the Agent alone, wherein the AUC is calculated over the first 60 minutes following introduction to an aqueous medium at pH 6.5 and 37° C.

In a further embodiment of the present invention, the composition retains substantially all of the Agent in solution following an increase in pH from a pH similar to that found in the stomach (for example pH 1.5 to 3.5) to a pH similar to that found in the upper intestine (for example pH 4.5 to 8). As mentioned above the Agent is readily soluble in the low pH environment of the stomach. We have found that the compositions according to the invention act to retain substantially all of the Agent initially dissolved in the low pH environment in solution when the pH is increased from about 1.5 to about pH 6.5 using the in-vitro pH shift test described in the Examples for at least 30 minutes, more preferably at least 60 minutes following shift to pH 6.5. Generally the compositions according the invention retain at least 60%, particularly 70% more particularly at least 80% and still more particularly at least 90% by weight of the Agent in solution for at least 30 minutes, more preferably at least 60 minutes following increase in pH to 6.5 in the pH shift dissolution test described herein.

In preferred embodiments the composition according the invention retain substantially all of the Agent in solution following a shift in pH from low pH to high pH (for example from 1.5 to 6.5) and provide enhanced solubilisation of the Agent, particularly at pH values similar to those found in the upper GI tract (for example pH 4.5 to 8, particularly pH 6.5).

Water-Soluble Cellulose Ethers and Esters of Water-Soluble Cellulose Ethers

By “cellulose ether” is meant an ether formed by the conversion of one or more hydroxy groups present on one or more of the anhydroglucose repeat unit of a cellulose polymer to provide one or more ether linked group on the cellulose polymer. By way of example, suitable ether linked groups that may be present on anhydroglucose repeat units of the cellulose polymer include (1-4C)alkyl optionally substituted by one or more substituent(s) selected from hydroxy, carboxy, (1-4C)alkoxy and hydroxy(1-4C)alkoxy. Particular ether linked groups include, for example, (1-4C)alkyl such as methyl or ethyl; hydroxy(1-4C)alkyl, such as 2-hydroxyethyl, 2-hydroxypropyl or 3-hydroxypropyl; (1-4C)alkoxy(1-4C)alkyl, such as 2-methoxyethyl, 3-methoxypropyl, 2-methoxypropyl or 2-ethoxyethyl; hydroxy(1-4C)alkoxy(1-4C)alkyl such as 2-(2-hydroxyethoxy)ethyl or 2-(2-hydroxypropoxy)propyl; carboxy(1-4C)alkyl, such as carboxymethyl; or groups of the formula H—[O-(1-4C)alkyl-]ₘ, wherein m=1 to 5, for example 1, 2 or 3, such as H—[O—CH(CH₃)CH₂—]ₘ or H—[O—CH₂CH₂—]ₘ. For the avoidance of any doubt, the term “ether-linked groups” refers to one or more of the above groups linked to the cellulose polymer by an oxygen atom. For example where the ether linked group is methyl, one or more of the hydroxy groups of the anhydroglucose repeat units are converted to methoxy.

The water-soluble cellulose ether may carry the same ether linked groups, for example methyl groups as in the case of methylcellulose. Alternatively the water-soluble cellulose ether may carry a plurality of different ether-linked groups. For example hydroxypropyl methylcellulose refers to a cellulose which carries both methyl and hydroxypropyl (for example 2-hydroxypropyl) ether linked groups. Water-soluble cellulose ethers carrying hydroxyethyl or hydroxypropyl ether-linked groups are often prepared by reacting cellulose with ethylene or propylene oxide to introduce the required ether groups. This process can result in a plurality of oxyethylene or oxypropylene chains of the formula H—[O—CH(CH₃)CH₂—]ₘ or H—[O—CH₂CH₂—]ₘ, wherein m is for example 1 to 5, linked to the cellulose backbone by an oxygen atom. Such groups are suitable for use in the present invention provided that the resulting cellulose ether is water-soluble.

By the term “water-soluble cellulose ether” is meant cellulose ethers that dissolve or disperse in water to give a colloidal solution or dispersion at a temperature of less than 30° C. (for example from 10 to 20° C.). Generally the water-soluble cellulose ethers will have a solubility in water of at least 20 mg/ml, suitably at least 30 mg/ml at a temperature of 10 to 20° C. (wherein the solubility is determined in un-buffered distilled water). Preferably the water-soluble cellulose ether has a high dissolution rate in water. Accordingly it is preferred that the solubility of the water-soluble ether described above is determined 2 hours, more preferably 1 hour, after adding the water-soluble cellulose ether to water. Suitable water-soluble cellulose ethers include, those listed in the Handbook of Pharmaceutical Excipients, 4^{th} Edition American Pharmaceutical Association, for example methylcellulose, hydroxyethylcellulose, hydroxypropylcellulose, hydroxypropyl methylcellulose, hydroxybutyl methylcellulose, hydroxyethyl ethylcellulose, a water-soluble salt of carboxymethylcellulose (for example sodium carboxymethylcellulose) and a water-soluble salt of carboxymethyl hydroxyethyl cellulose (for example sodium carboxymethyl hydroxyethylcellulose)

In one embodiment of the invention the suitable water-soluble cellulose ether is selected from, for example, methyl cellulose, hydroxyethylcellulose, hydroxypropylcellulose and hydroxypropyl methylcellulose.

In one embodiment of the present invention the water-soluble cellulose ether is hydroxypropyl methylcellulose (HPMC). As mentioned hereinbefore the cellulose polymer backbone of HPMC carried both methoxy and hydroxypropoxy (particularly 2-hydroxypropoxy) groups. A wide range of grades of HPMC may be used for example with a dynamic viscosity of up to 100,000 cP, provided that sufficient HPMC will dissolve at gastric pH levels to inhibit precipitation of the Agent upon an increase in pH. Preferably, the HPMC is a low viscosity grade, for example with a dynamic viscosity of 1000 cP or less, particularly 100 cP or less, still more particularly <60 cP, such as from 2 to 18, suitably from 5 to 7 cP, wherein the dynamic viscosity is measured in a 2% w/v aqueous solution of the HPMC at 20° C. The HPMC suitably has a degree of substitution of from 10 to 35% (suitably from 25 to 35%) methoxy groups and 2-30% (for example 3-30%, suitably from 5 to 15%) hydroxypropoxy groups. Unless specified otherwise the term “% degree of substitution” used herein refers to the average % by weight of methoxy and hydroxypropoxy groups based upon the dry weight of the water-soluble cellulose ether (eg HPMC). Particular grades of HPMC include 2910, 1828, 2208 and 2906 (wherein the first two digits refer to the average degree of methoxy substitution and the second two digits to the average degree of hydroxypropoxy substitution) as described in the Handbook of Pharmaceutical Excipients, 3^{rd} Edition 2000 American Pharmaceutical Association p 252. More particularly, the above grades of HPMC with a dynamic viscosity of from 2 to 18 cP (for example from 5 to 7 cP). Still more particularly the HPMC is grade 2910 with a dynamic viscosity of from 5 to 7 cP, wherein the dynamic viscosity is measured in a 2% w/v aqueous solution of the HPMC at 20° C.

Unless specified otherwise, the term dynamic viscosity used herein refers to viscosity measurement at the temperature quoted using a suitable apparatus such as a Brookfield viscometer fitted with a #2 spindle and a rotation rate of 60 rpm.

In a further embodiment of the present invention the water-soluble cellulose ether is selected from hydroxyethylcellulose and hydroxypropylcellulose. When the water-soluble cellulose ether is a hydroxypropylcellulose the degree of substitution is suitably more than 16%, for example 20 to 80%, particularly 20 to 40%. Suitably the hydroxypropylcellulose has a dynamic viscosity of from about 2 to about 30000 cP. Preferably, however the hydroxypropylcellulose has a low dynamic viscosity of for example 100 to 600 cP (such as from 150 to 450 cP), wherein the viscosity is measured in a 2% w/v aqueous solution of the hydroxypropylcellulose at 25° C. Alternatively the hydroxypropylcellulose may have a degree of substitution of from approximately 5 to approximately 16% hydroxypropoxy groups. Such hydroxypropyl celluloses are commercially available as “low substituted” hydroxypropyl cellulose. Although low substituted hydroxypropylcellulose is often described as water-insoluble, we have surprisingly found that low substituted hydroxypropylcellulose is sufficiently hydrophilic to prevent precipitation of the Agent from solution, and for the purposes of this invention low-substituted hydroxypropylcellulose is to be considered a water-soluble cellulose ether. When the cellulose ether is hydroxyethylcellulose it is suitably a water-soluble hydroxyethylcellulose with a molecular weight of for example, 85000 to 1,300,000, particularly 150,000 to 350,000 such as approximately 220,000 to 270,000. Generally, suitable water-soluble hydroxyethylcelluloses include those with a dynamic viscosity of from about 10 to about 10000 cP, particularly low viscosity hydroxyethylcellulose for example with a dynamic viscosity of from 50 to 250 cP, such as 80 to 125 cP, wherein the viscosity is measured in a 2% w/v aqueous solution of the hydroxyethylcellulose at 25° C. Suitably the hydroxyethylcellulose has a degree of substitution of approximately 0.8 to 2.5, such as from 0.8 to 1.5, for example approximately 1, wherein here degree of substitution refers to the average number of hydroxyethyl groups per anyhydroglucose ring of the cellulose.

In a further embodiment the water-soluble cellulose ether is methylcellulose, particularly a methylcellulose with a dynamic viscosity of from about 4 to about 15000 cP, particularly low viscosity methylcellulose with a viscosity of 5 to 100 cP, such as 10 to 25 cP, (25 w/v solution in water at 20° C. measured using for example an Ubbelohde viscometer in accordance with ASTM D2363). Suitably the methylcellulose has a degree of substitution of from 1 to 2, for example from 1.64 to 1.92, such as approximately 1.8, wherein here degree of substitution refers to the average number of methoxy groups per anyhydroglucose ring of the cellulose. Suitably the methylcellulose has a molecular weight of approximately 10,000 to 140,000, particularly approximately 10,000 to 50,000, for example 10,000 to 35,000. Suitable water-soluble methyl cellulose is commercially available, for example under the trade name Methocel™ such as Methocel™ A and Methocel™ MC ex Dow Inc.

By “ester of a water-soluble cellulose ether” used herein is meant an ester formed between one or more hydroxyl group(s) present in a water-soluble cellulose ether and one or more suitable organic acid(s) or reactive derivatives thereof, to thereby form ester linked groups on the water-soluble cellulose ether. Therefore the esters of water-soluble cellulose ethers carry both ester linked groups and ether linked groups on the cellulose polymer backbone. The ester of the water-soluble cellulose may carry the same ester linked moieties, for example acetate groups as in the case of HPMC acetate. Alternatively the ester of a water-soluble cellulose ether may carry a plurality of different ester-linked moieties (for example 2 or more moieties such as, for example, succinate and phthalate groups). For example HPMC acetate succinate refers to a mixed ester of HPMC which carries both succinate and acetate groups and HPMC acetate succinate trimellitate is a mixed ester of HPMC which carries acetate, succinate and trimellitate groups.

In a particular embodiment of the present invention the ester of a water-soluble cellulose ether is an ester of HPMC or hydroxypropylcellulose (HPC) which carries one or more ester groups selected from acetate, succinate, phthalate, isophthalate, terephthalate and trimellitate. Particular examples of esters of water-soluble cellulose ethers include, but is not limited to, HPMC acetate, HMPC succinate, HPMC acetate succinate, HPMC phthalate (commercially available as, for example HP-55 and HP 55-S), HPMC trimellitate, HPMC acetate phthalate, HPMC acetate trimellitate, HPC acetate phthalate, HPC butyrate phthalate, HPC acetate phthalate, succinate and HPC acetate trimellitate succinate. More particularly the ester of water-soluble cellulose ether is selected from HPMC acetate succinate (commercially available as Aqoat ex Shin-Etsu Chemical Co. for example Aqoat AS-LG).

In another embodiment of the invention, the water-soluble cellulose ether or ester of a water-soluble cellulose ether is selected from hydroxypropylcellulose, HPMC, hydroxyethylcellulose, methylcellulose, an ester of HPMC and an ester of hydroxypropyl cellulose, wherein said ester carries one or more ester groups selected from acetate, succinate, phthalate, isophthalate, terephthalate and trimellitate. In this embodiment a particular water-soluble cellulose ether and/or ester of a water-soluble cellulose ether is selected from hydroxypropyl cellulose, HPMC, hydroxyethylcellulose, methylcellulose and HPMC acetate succinate. More particularly, the water-soluble cellulose ether and/or ester of a water-soluble cellulose ether is selected from hydroxypropyl cellulose, hydroxyethylcellulose, methylcellulose and HPMC acetate succinate. In a further embodiment of the invention the water-soluble cellulose ether and/or ester of a water-soluble cellulose ether is selected from HPMC, hydroxypropyl cellulose, hydroxyethylcellulose, methylcellulose and HPMC acetate succinate. Still more particularly the pharmaceutical composition according to the present invention comprises the Agent, a water-soluble acid and a water-soluble cellulose ether selected from hydroxypropyl cellulose, hydroxyethylcellulose and methylcellulose. A particular water-soluble cellulose ether is hydroxypropylcellulose, more particularly a water-soluble hydroxypropylcellulose having a degree of substitution of more than 16%. Another particularly suitable water-soluble cellulose ether in this embodiment is methylcellulose. Another embodiment of the invention comprises the Agent, a water-soluble acid and a water-soluble cellulose ether selected from HPMC and methylcellulose. Suitable grades of the above mentioned water-soluble cellulose ethers are as hereinbefore described.

The composition according to the present invention may contain a single water-soluble cellulose ether or ester of a water-soluble cellulose ether or two or more such polymers.

The water-soluble cellulose ether, or ester of water-soluble cellulose ether is suitably present in the composition over a wide range for example from 2% to 70%, such as 2 to 50% or 10 to 40% by weight of the composition. Suitably, the weight ratio of the Agent to water-soluble cellulose ether, or ester of water-soluble cellulose ether is from 50:1 to 1:5, for example from 35:1 to 1:1, more particularly from 40:1 to 2:1, still more particularly from 33:1 to 2:1 such as from 33:1 to 10:1. In a further embodiment the weight ratio of Agent to cellulose ether or ester thereof is up to 32:1, for example from 32:1 to 1:1, more particularly from 30:1 to 2:1, still more particularly from 25:1 to 3:1.

In another embodiment of the invention the weight ratio of Agent to water-soluble cellulose ether, or ester of water-soluble cellulose ether is from 40:1 to 2.5:1, particularly from 30:1 to 3:1 and especially from 5:1 to 3:1. We have found that, in general, increasing the amount of cellulose ether or ester thereof such that the weight ratio of Agent to cellulose ether or ester thereof is less than about 3:1 (for example 1:1) gives only a relatively small further reduction in the rate of precipitation of the Agent, beyond that observed when the weight ratio of Agent to cellulose is approximately 3:1.

Acid

The acid is a water-soluble acid which may be an organic or inorganic acid. It is preferred however, that the acid is a water-soluble organic acid as we have found that certain water-soluble organic acids provide a particularly enhanced concentration of the Agent at a pH similar to that of the major absorption sites in the GI tract. The term “water-soluble” used herein in relation to the acid present in the composition refers to an acid that has a solubility of at least 0.2% by weight in water at 25° C.

Particularly, the acid has at least one pKa value which is at least one (preferably at least two) pK unit lower than the highest pKa of the basic groups present in the Agent. More particularly the acid has a pKa value which is at least one (preferably at least two) pK unit lower than the lowest pKa of the basic groups present in the Agent. By way of an example, in the case of the Agent, which has pKa values of approximately 5.3 and 7.2 the acid preferably has a pKa of less than 6.2, more preferably less than 4.3, still more preferably less than 3.3. In an embodiment of the invention the water-soluble acid is a water-soluble acid which is solid at ambient temperature (18-25° C.). The use of solid water-soluble acids is particularly convenient for the manufacture of compositions according to the invention, which compositions are in the form of a solid unit dosage form.

Particular inorganic acids include, water-soluble inorganic acids that are solid at ambient temperature, for example sulfamic acid.

Suitable organic acids are water-soluble organic molecules containing one or more acidic group, particularly compounds containing acidic groups selected from carboxylic and sulfonic acid groups, particularly those which are solid at ambient temperature.

Particular water-soluble organic acids include a water-soluble organic acid selected from a mono, di- or polybasic carboxylic acid and a mono, di or tri-sulfonic acid, preferably those which are solid at ambient temperature. Particular solid water-soluble carboxylic acids include, for example aliphatic mono or poly-carboxylic acids such as those containing from 2 to 8 carbon atoms, particularly from 2 to 6 carbon atoms, more particularly di-or tricarboxylic acids containing from 4 to 6 and especially 4 carbon atoms, any of which acids may be saturated or unsaturated. Examples of suitable solid water-soluble aliphatic mono-carboxylic acids include sorbic acid (2,4-hexandienoic acid). Examples of suitable solid water-soluble aliphatic di-carboxylic acids include adipic, malonic, succinic, glutaric, maleic or fumaric acid. The aliphatic carboxylic acid may be optionally substituted by one or more groups (for example 1, 2 or 3), which may be the same or different, selected from carboxy, amino and hydroxy. Suitable substituted solid water-soluble aliphatic carboxylic acids include for example hydroxy substituted aliphatic mono-carboxylic acids such as gluconic acid, solid forms of lactic acid, glycolic acid or ascorbic acid; hydroxy substituted aliphatic di-carboxylic acids such as malic, tartaric, tartronic (hydroxymalonic), or mucic (galactaric) acid; hydroxy substituted aliphatic tri-carboxylic acids, for example citric acid; or amino acids carrying an acidic side chain, such as glutamic acid or aspartic acid.

Suitable aromatic carboxylic acids include water-soluble aryl carboxylic acids containing up to 14 carbon atoms. Suitable aryl carboxylic acids comprise an aryl group, for example a phenyl or naphthyl group which carries one or more carboxyl groups (for example 1, 2 or 3 carboxy groups). The aryl group is optionally substituted by one or more groups (for example 1, 2 or 3), which may be the same or different selected from hydroxy, (1-4C)alkoxy (for example methoxy) and sulfonyl. Suitable examples of aryl carboxylic acids include, for example benzoic, phthalic, isophthalic, terephthalic or trimellitic acid (1,2,4-benzenetricarboxylic acid). A preferred water-soluble aromatic carboxylic acid is benzoic acid.

A single water-soluble acid may be used or a combination of two or more such acids. When the water-soluble acid is a carboxylic acid, the acid may be incorporated into the composition as, for example a suitable carboxylic acid derivative, for example as an anhydrate or as a lactone, provided that the carboxylic acid derivative provides an acidic pH within the composition when it exposed to an aqueous medium.

In a particular embodiment of the invention the acid is a solid water-soluble aliphatic polycarboxylic acid (preferably a di- or tri-carboxylic acid) with 4 to 6 carbon atoms, which acid is saturated or unsaturated and which is optionally substituted by one or more hydroxy groups. Particular acids in this embodiment are selected from maleic, fumaric, malic, succinic, tartaric and citric acid. It is particularly preferred that the water-soluble acid is selected from malic, maleic and succinic acid. In a further embodiment of the invention the water-soluble acid is selected from fumaric acid and malic acid.

Generally, the composition may contain a molar ratio of Agent to acid of from 10:1 to 1:10, for example from 2:1 to about 1:10. In embodiments an equimolar or, preferably, a molar excess of the acid is present, for example a molar ratio of Agent to acid of from 1:1 to 1:10, particularly from 1:2 to 1:7, more particularly from 1:3 to 1:6.

Without wishing to be bound by theory it is thought that the acid present in the composition does not enhance dissolution of the Agent simply by decreasing the pH of the bulk liquid into which the composition according the invention is placed. Instead it is thought that the combined effect of the acid and the water-soluble cellulose ether (or ester thereof) in close contact with the Agent in the composition provides an enhanced solubilisation and inhibition of precipitation of the Agent by way of an interaction between the Agent, acid and water-soluble cellulose ether. The composition of the invention thereby provides an increased concentration of the Agent following oral administration to a patient compared to administration of the Agent alone.

The Agent

Typically the Agent will be present in an amount within the range of from 1 to 99%, and suitably from 1 to 70%, for example from 5 to 65% and especially from 10 to 60% by weight of the composition.

Suitably a unit dose of the composition according to the invention may contain from 0.01 mg to 1 g of Agent. Suitably a unit dose of the composition will contain a daily dose of the Agent in a quantity sufficient to provide the desired therapeutic benefit. Suitable quantities of the Agent in unit doses in different embodiments include, for example 10, 15, 25, 50, 75, 100, 125, 150, 200, 250, 300, 350, 400, 450, 500, 550, 600, 650, 700, 750, 800, 850, 900, 950, 1000 mg, or higher depending upon the dose required and the particular form of the pharmaceutical composition. In an embodiment a unit dose of the composition contains 100, 150, 250 or 500 mg of the Agent, especially 250 mg of the Agent.

The Agent may be used in the free base form or as a pharmaceutically acceptable salt of the compound of formula I, such as a pharmaceutically acceptable mono- or di-acid addition salt with, for example, a suitable inorganic or organic acid, for example selected from hydrochloric, hydrobromic, sulfuric, phosophoric, trifluoroacetic, citric, tartaric, fumaric, maleic, methane sulfonic or 4-toluene sulfonic acid. Accordingly references herein to the Agent are, unless stated otherwise, intended to cover the free base form of the Agent or a pharmaceutically acceptable salt thereof. In one embodiment, the Agent is in the free base form, particularly a crystalline free-base form. As will be clear, the term “free-base form” refers to the case when the Agent is not in the form of a salt. In embodiments the Agent may be used in an amorphous form, a crystalline form or as a mixture of amorphous and crystalline forms.

In view of the foregoing, a particular pharmaceutical composition according to the present invention comprises:

(i) the Agent;

(ii) a water-soluble cellulose ether (particularly selected from methyl cellulose, hydroxyethylcellulose, hydroxypropylcellulose and hydroxypropyl methylcellulose, more particularly selected from methylcellulose and hydroxypropyl methylcellulose); and

(iii) a water-soluble organic acid which is solid at ambient temperature (particularly an organic acid selected from an aliphatic polycarboxylic acid (preferably a di- or tri-carboxylic acid) with 4 to 6 carbon atoms, which acid is saturated or unsaturated and which is optionally substituted by one or more hydroxy groups (particularly maleic, fumaric, malic, succinic, tartaric or citric acid, more particularly malic, maleic or succinic acid, and especially fumaric acid or malic acid).

In a further embodiment the composition according to the present invention comprises:

(i) from 10 to 60 (particularly 30 to 50) parts of the Agent;

(ii) from 2 to 70 (particularly 5 to 40) parts of a water-soluble cellulose ether (particularly selected from methyl cellulose, hydroxyethylcellulose, hydroxypropylcellulose and hydroxypropyl methylcellulose, more particularly selected from methyl cellulose and hydroxypropyl methylcellulose); and

(iii) from 10 to 70 (particularly 30 to 60) parts of a water-soluble organic acid which is solid at ambient temperature (particularly an organic acid selected from an aliphatic polycarboxylic acid (preferably a di- or tri-carboxylic acid) with 4 to 6 carbon atoms, which is saturated or unsaturated and which is optionally substituted by one or more hydroxy groups (particularly maleic, fumaric, malic, succinic, tartaric or citric acid, more particularly malic, maleic or succinic acid, and especially fumaric acid or malic acid);

wherein all parts are by weight and the sum of the parts (i)+(ii)+(iii)=100.

In these embodiments the molar ratio of Agent to organic acid is suitably as described above, for example, from 1:2 to 1:7, more particularly from 1:3 to 1:6. The weight ratio of Agent to water-soluble cellulose ether/ester thereof is suitably more than approximately 3:1, for example from about 30:1 to about 3:1 and especially from about 5:1 to 3:1.

In a further embodiment the composition according to the present invention comprises:

(i) from 10 to 60 (particularly 30 to 50) parts of the Agent;

(ii) from 2 to 70 (particularly 5 to 40) parts of a water-soluble cellulose ether selected from methyl cellulose and hydroxypropyl methylcellulose; and

(iii) from 10 to 70 (particularly 30 to 60) parts of a water-soluble organic acid selected from fumaric acid and malic acid;

wherein all parts are by weight and the sum of the parts (i)+(ii)+(iii)=100;

and wherein the molar ratio of Agent to organic acid is from 1:3 to 1:6.

Formulation

The composition according to the present invention may be prepared as a simple physical mixture wherein the Agent, acid and water-soluble cellulose ether or ester thereof are mixed together as a dry mixture of the components of the composition. In another embodiment the composition according to the present invention may be prepared as a wet granulate, for example by wet granulating a physical mixture of the components of the composition of the invention. In an alternative embodiment the composition according to the present invention may be prepared as a solid dispersion formulation. A particular solid dispersion formulation comprises a solid dispersion of the Agent in a matrix formed by the water-soluble cellulose ether or ester of the water-soluble cellulose ether together with the acid.

Optionally additional excipients may be included in the composition according to the present invention. Additional excipients which may be present include for example, one or more fillers (diluents), binders, disintegrants or lubricants. The composition according to the present invention may also include other excipients commonly used in pharmaceutical compositions including, for example preservatives, stabilisers, anti-oxidants, silica flow conditioners, antiadherents, wetting agents or glidants. The optional additional excipients that may be used are well known to those skilled in the art and are described in, for example the Handbook of Pharmaceutical Excipients, 4^{th} Edition, American Pharmaceutical Association; The Theory and Practice of Industrial Pharmacy, 3^{rd} Edition, Lachman et al. 1986; Pharmaceutical Dosage Forms: Tablets Volume 1, 2^{nd} Edition, Lieberman, Hebert A., et al, 1989; Modern Pharmaceutics, Banker, Gilbert and Rhodes, Christopher T, 3^{rd} edition, 1995; and Remington's Pharmaceutical Sciences, 20^{th} Edition, 2000.

Suitable fillers include, for example, lactose (which may be in an anhydrous or hydrated form, for example lactose monohydrate), sugar, starches (for example corn, wheat, maize, potato), modified starches (for example as starch hydrolysates or pregelatinized starch which may be thermally, mechanically or chemically modified), microcrystalline starches, mannitol, sorbitol, trehalose, maltose, inorganic salts (e.g. calcium carbonate, magnesium carbonate, dibasic calcium phosphate (anhydrous/dihydrate), tribasic calcium phosphate), cellulose, cellulose derivatives (e.g. microcrystalline cellulose), calcium sulfate, xylitol and lactitol.

Suitable binders include, for example, polyvinylpyrrolidone (for example povidone K25-32, particularly K29-32, wherein the “K value” is an indication of the average molecular weight range obtained from the Fikentscher equation described in the Handbook of Pharmaceutical Excipients, 3^{rd} Edition 2000 American Pharmaceutical Association p 433), lactose (which may be in an anhydrous or hydrated form, for example lactose monohydrate), starches, modified starches, sugars, gum acacia, gum tragacanth, guar gum, pectin, wax binders, microcrystalline cellulose, methylcellulose, carboxymethylcellulose and salts thereof (for example sodium carboxymethylcellulose), hydroxypropyl methylcellulose, hydroxyethyl cellulose, hydroxypropyl cellulose, copolyvidone, gelatin and alginates (for example sodium alginate).

Suitable disintegrants include, for example, croscarmellose sodium, crospovidone, polyvinylpyrtolidone, sodium starch glycolate, starches, microcrystalline cellulose carboxymethylcellulose and salts thereof (for example sodium or calcium carboxymethylcellulose), hydroxypropyl methylcellulose, hydroxypropylcellulose (particularly low substituted hydroxypropylcellulose i.e. hydroxypropylcellulose containing approximately 5 to 16% by weight hydroxypropoxy groups) or alginic acid.

Suitable lubricants include, for example, magnesium stearate, stearic acid, palmitic acid, calcium stearate, talc, carnauba wax, hydrogenated vegetable oils, mineral oil, polyethylene glycols, sodium lauryl sulfate and sodium stearyl fumarate.

Wetting agent(s) may be incorporated into the composition, for example to aid formulation of the composition. The wetting agents may also provide some additional solubilisation of the Agent, however, the presence of a wetting agent is not essential for solubilisation of the Agent in the compositions according to the present invention. Suitable wetting agents include pharmaceutically acceptable surfactants which may be ionic or non-ionic. Particular non-ionic surfactants include, for example, polyethylene glycols, polyoxyethylene esters and ethers, for example polyethoxylated castor oil (for example Cremophor EL), polyethoxylated hydrogenated castor oil, polyethoxylated fatty acids derived from castor oil, or polyethoxylated fatty acids derived from hydrogenated castor oil; ethoxylated stearic acid, for example Solutol HS15; and ethylene oxide-propylene oxide copolymers, for example ethylene oxide-propylene oxide block copolymers, such as Pluronic or Tetronic surfactants.

Suitable pharmaceutically acceptable ionic surfactants may be anionic, cationic or zwitterionic. Suitable anionic surfactants include, for example, sodium dodecyl sulfate (sodium lauryl sulfate), potassium myristate, sodium laurate or sodium lauryl sulfonate; di-alkyl sulfoscuccinates, such as dioctyl sulfosuccinates (e.g. Docusate sodium or Aerosol OT) or sodium diamyl sulfosuccinate (Aerosol AY). Suitable cationic surfactants include for example quaternary ammonium compounds such as for example cetyltrimethylammonium bromide (Cetramide), trimethyltetradecylammonium bromide (Myristamide), lauryl trimethylammonium bromide (Lauramide) or benzalkonium chloride.

Suitably the composition contains less than 5%, more particularly less than 2% by weight wetting agent, for example from 0.1 to 1.5% by weight wetting agent.

Suitably one or more fillers will be present in an amount of from 1 to 90% by weight, particularly 10 to 90% by weight, for example from 10 to 50%, particularly 30 to 50% by weight.

Suitably one or more binders will be present in an amount of from 0.5 to 50% by weight, for example from 0.5 to 10% by weight.

Suitably one or more disintegrants will be present in an amount of from 0.5 to 25%, particularly 0.5 to 20%, for example from 1 to 10% by weight.

Suitably one or more lubricants will be present in an amount of from 0.1 to 5% by weight, for example from 0.5 to 3% by weight.

It will be appreciated that a particular excipient may act as a both a binder and a filler, or as a binder, filler and disintegrant. Typically the combined amount of filler, binder and disintegrant comprises, for example from 2 to 85% by weight of the composition. For example in one embodiment of the invention the combined amount of filler, binder and disintegrant comprises from 2 to 40% by weight of the composition. In another embodiment the combined amount of filler, binder and disintegrant comprises 40 to 80% by weight of the composition.

A further embodiment of the invention provides an immediate release pharmaceutical composition comprising the Agent, a water-soluble acid (preferably a water-soluble organic acid), a water-soluble cellulose ether and/or ester of a water-soluble cellulose ether and one or more excipients selected from fillers, binders, disintegrants and lubricants. A still further embodiment of the invention provides a solid pharmaceutical composition for oral administration comprising the Agent, a water-soluble organic acid, a water-soluble cellulose ether, one or more fillers, one or more binders, one or more disintegrants and one or more lubricants.

Physical Mixtures

The pharmaceutical composition of the invention may be prepared as a physical mixture using standard techniques and manufacturing processes generally known in the art, for example by dry blending or as a granulate prepared by wet granulation of the components. The resulting blend/granules may then be used to prepare a number of dosage forms as discussed below.

Suitable wet granulation techniques are well known in the art, for example as described in Pharmaceutics, The Science of Dosage Form Design, edited by Aulton, Churchill Livingstone, 2002. For example, a suitable wet granulation technique comprises, blending together the Agent, the acid, the water-soluble cellulose ether and/or ester of a water-soluble cellulose ether and optionally other excipients if desired, using, for example a granulator. The resulting powder blend is then granulated with a small volume of granulation liquid. Preferably the granulation liquid does not contain any wetting agent(s). A suitable granulation liquid is purified water. The resulting granulate is then passed through a screen, to break up large aggregates, dried and passed though a mill. Suitable additional excipients may then be added to the granules (for example filler, disintegrant, wetting agent, binder and lubricant) and after blending the resultant homogeneous granule mixture may then be used to prepare a dosage form, for example by compressing the granules into a tablet or filling into a suitable capsule to provide a capsule formulation.

A suitable dry blending technique comprises for example, blending together the Agent, the acid and the water-soluble cellulose ether and/or ester of a water-soluble cellulose ether and optionally wetting agent, one or more fillers, one or more binders and one or more disintegrants, as well as other additional excipients if desired. The components of the blend prior to blending, or the blend itself, may be passed through a mesh screen, for example a 400-700 μm mesh screen. A lubricant, which may also be screened, is then added to the blend and blending continued until a homogeneous mixture is obtained. The resulting blend may then processed using conventional techniques to provide the final dosage form such as a tablet or granule formulation.

It will be appreciated that modifications of the dry blending and wet granulation techniques, including the order of addition of the components and their screening and blending prior to compression into tablets, may be carried out according to principles well known in the art.

Solid Dispersions The composition according to the invention may be prepared as a solid dispersion of the Agent in a matrix. The term “solid dispersion” refers to a dispersion of the Agent in the matrix. The Agent may be present in the matrix as drug rich domains dispersed in the matrix. The solid dispersion may also comprise some of the Agent dissolved in the matrix. The matrix of the solid dispersion may be formed by the water-soluble acid; the water-soluble cellulose ether or ester of a water-soluble cellulose ether; or both the water-soluble acid and the water-soluble cellulose ether or ester of a water-soluble cellulose ether.

Methods for preparing solid dispersions are known in the art, including mechanical processes such as milling or extrusion, thermal processing such as melt extrusion, and solvent processing such as solvent precipitation and solvent evaporation. These standard processes are well known to those skilled in the art, for example as described in the Encyclopaedia of Pharmaceutical Technology, published by Dekker Inc, Vol. 3, 337-352 (Vadnere). In the present invention a particularly suitable method comprises the steps of dissolving the Agent and the water-soluble cellulose ether or ester of a water-soluble cellulose ether in a common solvent and removing the solvent. The solvent can be routinely selected according to the particular cellulose used and the preparation method adopted. The solvent is selected to dissolve both the Agent and the water-soluble cellulose ether or ester thereof. Methods for removing the solvent from the solution include evaporation techniques such as rotary evaporation, spray drying, lyophilisation and thin film evaporation. Other solvent removal methods which may be used include solvent controlled precipitation (for example addition of a suitable anti-solvent for the Agent/cellulose), pH controlled precipitation, spray congealing, spray drying, melt extrusion and supercritical fluid technology. The acid is preferably incorporated into the solid dispersion by including the acid in the initial solution of the Agent, cellulose and solvent to give a solid dispersion of the acid and Agent in the cellulose. Alternatively, the acid may be incorporated after forming the solid dispersion of the Agent in the cellulose ether by simply mixing the acid with the solid dispersion. Similarly, in those embodiments where the matrix of the solid dispersion is provided by the water-soluble acid, the water-soluble cellulose ether or ester thereof may be incorporated by mixing the cellulose with the Agent/water-soluble acid solid dispersion.

The solid dispersion of the Agent in the water-soluble cellulose ether or ester of a water-soluble cellulose ether together with the acid may contain additional excipients or be formulated with additional excipients such as those described above to give the required final dosage form.

Thus a further aspect of the present invention provides a method of preparing a pharmaceutical composition which comprises admixing the Agent with a water-soluble acid and a water-soluble cellulose ether and/or or ester of a water-soluble cellulose ether and optionally other excipients, wherein the Agent, acid and water-soluble cellulose ether, ester of a water-soluble cellulose ether and optional excipients are as hereinbefore described in relation to the first aspect of the invention. Suitable methods for admixing the components of the composition are as hereinbefore described, particularly, dry blending, wet granulation or the preparation of a solid dispersion.

Dosage Form

The composition according to the present invention may be formulated to provide a variety of immediate release dosage forms, particularly solid immediate release dosage forms suitable for oral administration.

By the term “immediate release” used herein is meant that the solid dosage form releases substantially all of the Agent within 60 minutes (preferably within 30 minutes) following immersion in a pH 1.5 aqueous solution at 37° C. By “substantially all of the Agent” is meant that at least 70, 80, 90 or more particularly 95% of the Agent is released from the composition within 60 minutes (preferably within 30 minutes) following immersion in a pH 1.5 aqueous solution at 37° C. The Agent may be released from the composition as a dispersion into the dissolution medium or, preferably, quickly dissolves in the medium. The immediate release compositions according to the invention are expected to provide rapid dissolution of the Agent in the acid environment of the stomach where the solubility of the Agent is high and in the event that part of the composition does not dissolve in the stomach, for example as a result of rapid gastric emptying, the enhanced dissolution properties of the composition at higher pH provides for in-situ dissolution of the Agent in the upper intestine, thereby increasing the concentration of Agent at the major absorption site. Formulations suitable as immediate release compositions include for example, tablet, capsule, solid dispersion and (particularly) physical mixture and granulation formulations as described herein.

In a preferred embodiment the pharmaceutical composition of the invention is formulated into an oral immediate release dosage form, for example as a powder or granule mixture or as a suspension in a suitable liquid medium. Generally, however, the pharmaceutical composition is prepared as a solid immediate release dosage form suitable for oral administration, particularly a solid immediate release unit dosage form suitable for daily oral administration. Examples of suitable solid dosage forms include tablet, pellet, granule or capsule formulations.

When the pharmaceutical composition according to the invention is a solid immediate release dosage form such as a tablet, pellet or granules the solid composition optionally further comprises a suitable coating, for example a film coating. A coating can be used to provide protection against, for example, degradation by light or to colour the formulation. Suitable coatings, such as film coatings, that may be applied to the composition according to the invention comprise a film-forming agent, for example a sugar or conveniently, a water-soluble cellulose ether as hereinbefore described, particularly HPMC. The amount of film-forming agent used will depend upon the desired properties of the film coating and can be adjusted accordingly by those skilled in the art.

In a particular embodiment of the invention the pharmaceutical composition comprises a solid immediate release pharmaceutical composition (such as a tablet, pellet or granule formulation) comprising:

(i) a core comprising the Agent and the water-soluble acid (preferably a water-soluble organic acid); and

(ii) a coating comprising a water-soluble cellulose ether.

In this embodiment suitable water-soluble cellulose ethers are as hereinbefore described, especially hydroxypropyl methylcellulose (particularly grades, 1828, 2208, 2906 and especially 2910 having a dynamic viscosity of from 2 to 18 cP) or methylcellulose. Suitably the coating is applied as a film coating as herein described. The core comprising the Agent and the acid may comprise any of the compositions described hereinbefore containing a water-soluble cellulose ether or ester of a water-soluble cellulose ether (and optionally other additional excipients as hereinbefore described). Alternatively, the core may comprise the Agent and acid without a water-soluble cellulose ether or ester of a water-soluble cellulose ether. Accordingly, in this embodiment the water-soluble cellulose ether may be present entirely in the coating. Alternatively, water-soluble cellulose ether(s) may be present in both the core and the coating.

In a further embodiment of the present invention there is provided a solid immediate release pharmaceutical composition (such as a tablet, pellet or granule formulation) comprising (i) a core comprising the Agent and the acid; and (ii) a coating (particularly a film coating), wherein a water-soluble cellulose ether is present in at least one of the core or coating. In this embodiment the water-soluble cellulose ether may be present entirely in the coating, entirely in the core or be present in both the core and the coating.

When the composition is prepared as a capsule, a composition comprising the agent, water-soluble acid and water-soluble cellulose ether is first prepared as a physical mixture (for example a dry powder mixture or granule formulation) or a solid dispersion as described above. The composition containing the Agent is then filled into a capsule to provide a capsule formulation, suitable capsules are well known in the art. For example, hard gelatin, water-soluble cellulose ether (for example hydroxypropyl methylcellulose) and starch capsules. When the capsule contains a water-soluble cellulose ether, the capsule may be used to provide some or all of the water-soluble cellulose ether present in the composition according to the invention.

The Agent possesses anti-proliferative activity and accordingly the compositions according to the present invention are useful in the treatment of conditions such as those described in International Patent Application WO 96/33980. For example, the composition of the invention is useful for the treatment of many common human cancers such as lung (including small cell lung cancer and non small cell lung cancer), breast, prostate, ovarian, colorectal, gastric, brain (including glioma and pituitary adenoma), head and neck, bladder, pancreas, oesophageal, stomach, renal, skin (including malignant melanoma), gynaecological (including cervical, endometrial, vaginal, vulval and uterine) and thyroid cancer and in the treatment of a range of leukaemias, lymphoid malignancies and solid tumours such as carcinomas and sarcomas. It is further expected that the compositions of the invention will be useful for the treatment of other diseases involving excessive cellular proliferation such as benign skin hyperplasia, for example psoriasis, and benign prostatic hypertrophy (BPH).

The anti-proliferative properties of the Agent, particularly in the treatment of non-small-cell lung cancer, are well known in the art. Clinical trials of the Agent have been carried out and the Agent has shown clinical activity. Data from two large Phase II trials (IDEAL 1 and 2) in non-small-cell lung cancer showed that 250 mg/day Agent provided response rates of 11.8-18.4%, with the majority of adverse events generally being mild to moderate skin and gastrointestinal disorders (Fukuoka et al, (Multi-institutional randomized phase II trial of gefitinib for previously treated patients with advanced non-small-cell lung cancer; J. Clin Oncol 2003; 21: 2237-2246) and Kris et al (Efficacy of gefitinib, an inhibitor of the epidermal growth factor receptor tyrosine kinase, in symptomatic patients with non-small cell lung cancer. A randomized trial; JAMA 2003; 290: 2149-2158)). As mentioned hereinbefore the Agent has been approved in a number of countries for the treatment of non-small-cell lung cancer. This data supports the medicinal efficiency of the Agent, the active ingredient used in the pharmaceutical composition of the invention

A further aspect of the present invention provides a pharmaceutical composition according to the invention as hereinbefore defined for use as a medicament.

The Agent present in the compositions of the invention possesses anti-proliferative properties such as anti-cancer properties which are believed to arise from its erbB1 receptor tyrosine kinase inhibitory activity. Accordingly the composition of the invention is expected to be useful in the treatment of diseases or medical conditions mediated alone or in part by erbB1 receptor tyrosine kinases, i.e. the composition of the invention may be used to produce an erbB1 receptor tyrosine kinase inhibitory effect in a warm-blooded animal in need of such treatment. Thus the composition of the invention provides a method for treating the proliferation of malignant cells characterised by inhibition of erbB1 receptor tyrosine kinases, i.e. the composition of the invention may be used to produce an anti-proliferative effect mediated alone or in part by the inhibition of erbB1 receptor tyrosine kinase. Accordingly the active substance of the invention is expected to be useful in the treatment of psoriasis and/or cancer by providing an anti-proliferative effect, particularly in the treatment of erbB1 receptor tyrosine kinase sensitive cancers such as the cancers hereinbefore described.

In an embodiment of the invention there is provided, a pharmaceutical composition according to the invention as hereinbefore defined for use in producing an anti-proliferative effect in a warm-blooded animal (preferably a human). In another embodiment there is provided a pharmaceutical composition according to the invention as hereinbefore defined for use in the treatment of a cancer. In a still further embodiment there is provided a pharmaceutical composition according to the invention for use in the prevention or treatment of tumours which are sensitive to the inhibition of erbB1 receptor tyrosine kinase.

A further aspect of the present invention provides the use of a composition according to the invention as hereinbefore defined in the manufacture of a medicament for use in producing an anti-proliferative effect in a warm-blooded animal (preferably a human).

A further aspect of the present invention provides the use of a composition according to the invention as hereinbefore defined in the manufacture of a medicament for use in the treatment of a cancer.

A further aspect of the present invention provides a method for substantially pH independent delivery of the Agent to a patient in need of the Agent, comprising orally administering to said patient a composition according to the first aspect of the present invention as hereinbefore defined.

A further aspect of the present invention provides the use of a composition according to the first aspect of the invention as hereinbefore defined in the manufacture of a medicament for substantially pH independent delivery of the Agent to a warm blooded mammal (preferably a human). The term “substantially pH independent delivery of the Agent” refers to the provision of a high concentration of the Agent at the site of absorption of the Agent that is substantially independent of gastric pH and/or rate of gastric emptying. For example, as discussed hereinabove, the composition according to the invention inhibits precipitation of the Agent when the pH of a solution of the Agent is increased from a value similar to gastric pH to a pH similar to those found in the upper intestine. The composition of the invention also provides enhanced solubilisation of the Agent, particularly at higher pH (such as pH 4.5 to 8, particularly at pH of about 6.5). Therefore, in the event that the composition has not released all of the Agent in the stomach before the composition passes into the upper intestine (for example due to a high rate of gastric emptying), or where the gastric pH is high, it is expected that the enhanced solubilisation provided by the composition of the invention will provide an increased concentration of Agent in the upper intestine compared to administering the Agent alone.

The enhanced solubilisation and/or inhibition of precipitation of Agent from solution is expected to reduce the variability of exposure of the Agent in a single patient and between patients as a result of the substantial pH independent delivery of the Agent provided by the present invention.

Accordingly, a further aspect of the present invention provides a method for reducing inter-patient and/or intra-patient variability in bioavailability and/or plasma concentrations of the Agent in a patient in need of the Agent comprising orally administering to said patient a pharmaceutical composition according to the first aspect of the present invention as hereinbefore defined.

A further aspect of the present invention provides the use of a pharmaceutical composition according to the first aspect of the present invention as hereinbefore defined in the manufacture of a medicament for reducing inter-patient and/or intra-patient variability in bioavailability and/or plasma concentrations of the Agent.

A further aspect of the present invention provides the use of a water-soluble acid (preferably a water-soluble organic acid) and a water-soluble cellulose ether or an ester or a water-soluble cellulose ether in the manufacture of a medicament containing the Agent for reducing inter-patient variability in bioavailability andlor plasma concentrations of the Agent.

The dose of Agent required of the composition of the invention for the therapeutic or prophylactic treatment of a particular disease or medical condition (for example a proliferative disease) will necessarily be varied depending on for example, the host treated and the severity of the illness being treated. Preferably a daily dose of the Agent in the range, for example, 0.5 to 15 mg per kg body weight is received. More preferably a daily dose of the composition containing the Agent in the range, for example, 1 to 10 mg per kg body weight is received. A unit dose of the composition containing the Agent in the range, for example, 1 to 3000 mg, such as 1 to 1000 mg, conveniently 100 to 750 mg, more conveniently 200 to 600 mg, preferably about 250 mg is envisaged. Suitable ratios of Agent to water-soluble cellulose ether or ester of a water-soluble cellulose ether and Agent to water-soluble acid in such unit dosage forms are as hereinbefore defined.

According to a further aspect of the present invention there is provided the use of a water-soluble acid and a water-soluble cellulose ether or an ester of a water-soluble cellulose ether in the manufacture of a pharmaceutical composition comprising the Agent to increase the solubilisation of the Agent in an aqueous medium (preferably and aqueous medium with a pH value similar to that found in the upper GI tract of a human (for example pH 4.5 to 8)) compared to the solubilisation of the Agent alone in the same aqueous medium.

According to a further aspect of the aspect of the invention there is provided a method for increasing the solubilisation of the Agent in an aqueous medium (preferably with a pH value similar to those found in the upper GI tract of a human (for example pH 4.5 to 8)); comprising adding to said aqueous medium a pharmaceutical composition according to the first aspect of the invention as hereinbefore defined; wherein the solubilisation of the Agent from the composition is increased compared to the solubilisation of the Agent alone in the same aqueous medium.

According to a further aspect of the aspect of the invention there is provided a method for increasing the solubilisation of the Agent in an aqueous medium (preferably with a pH value similar to those found in the upper GI tract of a human (for example pH 4.5 to 8)); comprising preparing a pharmaceutical composition according to the first aspect of the invention as hereinbefore defined; wherein the solubilisation of the Agent from the pharmaceutical composition in said aqueous medium is increased compared to the solubilisation of the Agent alone in the same aqueous medium. Suitable pharmaceutical compositions and methods for the preparation thereof for use in this aspect of the invention are as described herein.

The enhanced solubilisation of the Agent provided by the methods and use according to these aspects of the invention may be determined as hereinbefore described, for example as measured by an increase in the Cmax or the AUC of the Agent following addition of the pharmaceutical composition to the aqueous medium.

In preferred embodiments of the invention, the use of a water-soluble cellulose ether or ester thereof together with a water-soluble acid in a composition containing the Agent provides a more than additive increase in the solubilisation of the Agent from the composition compared to any of: (i) the solubilisation of the Agent alone, (ii) the solubilisation of the Agent from a composition containing the Agent and the water-soluble cellulose ether or an ester thereof; or (iii) the solubilisation of the Agent from a composition containing the Agent and the water-soluble acid. In particular embodiments the use of a water-soluble cellulose ether or ester thereof together with a water-soluble acid in a composition containing the Agent provides a synergistic increase in the solubilisation of the Agent compared to any of: (i) the solubilisation of the Agent alone, (ii) the solubilisation of the Agent from a composition containing the Agent and the water-soluble cellulose ether or an ester thereof; or (iii) the solubilisation of the Agent from a composition containing the Agent and the water-soluble acid.

According to a further aspect of the present invention there is provided the use of a water-soluble acid, and a water-soluble cellulose ether or an ester of a water-soluble cellulose ether in the manufacture of a pharmaceutical composition comprising the Agent to inhibit the precipitation of the Agent from an aqueous solution.

According to a further aspect of the present invention there is provided a method for inhibiting the rate of precipitation of the Agent from an aqueous solution comprising adding to an aqueous medium with a low pH (for example similar to the gastric pH in a human, such as about pH 1.5) a pharmaceutical composition according to the first aspect of the invention as hereinbefore defined.

According to a further aspect of the present invention there is provided a method for inhibiting the rate of precipitation of the Agent from an aqueous solution comprising preparing a pharmaceutical composition according to the first aspect of the invention as hereinbefore defined. Suitable pharmaceutical compositions and methods for the preparation thereof for use in this aspect of the invention are as described herein.

These methods and uses according to these aspects of the invention are particularly suitable for inhibiting precipitation of the Agent following an increase in pH from a low pH (for example similar to that found in the stomach, such as about pH 1.5) to a pH similar to that found in the upper GI tract of a human, such as the small intestine (for example pH 4.5 to 8).

More particularly, the use and method according to these aspects of the present invention are expected to reduce the rate of precipitation of the Agent from an aqueous solution in-vivo as the Agent passes from the stomach of a patient to the higher pH regions of the GI tract from where the Agent is absorbed (which is thought to be the upper intestine). The degree of inhibition of precipitation of the Agent from solution is as hereinbefore defined in relation to the first aspect of the invention, for example as measured using the pH shift dissolution test described in the Examples.

In preferred embodiments of the invention, the use of a water-soluble cellulose ether or ester thereof together with a water-soluble acid in a composition containing the Agent provides a more than additive effect in reducing the rate of precipitation of the Agent from an aqueous solution following an increase in pH from a low pH (for example similar to that found in the stomach, such as about pH 1.5) to a pH similar to that found in the upper GI tract of a human, such as the small intestine (for example pH 4.5 to 8) compared to the use of any of: (i) the Agent alone, (ii) a composition containing the Agent and the water-soluble cellulose ether or an ester thereof; or (iii) a composition containing the Agent and the water-soluble acid. In particular embodiments the use of a water-soluble cellulose ether or ester thereof together with a water-soluble acid in a composition containing the Agent provides a synergistic effect on reducing the rate of precipitation of the Agent from an aqueous solution following an increase from low pH to a high pH compared to any of: (i) the Agent alone, (ii) a composition containing the Agent and the water-soluble cellulose ether or an ester thereof; or (iii) a composition containing the Agent and the water-soluble acid.

The aqueous solution/medium referred to in the above mentioned uses and methods of the invention may be an in-vitro aqueous medium, for example an aqueous medium in an in-vitro assay system or, particularly, the medium is an in-vivo aqueous medium, for example an in-vivo gastric or intestinal fluid.

Accordingly in an embodiment of the invention there is provided the use of a water-soluble acid and a water-soluble cellulose ether, or an ester of a water-soluble cellulose ether in the preparation of an immediate release pharmaceutical composition adapted for oral administration (suitably a solid immediate release pharmaceutical composition such as a tablet or capsule) to inhibit the rate of precipitation of the Agent from solution in-vivo following administration of the pharmaceutical composition to a warm-blooded animal such as a human.

In preferred embodiments of the invention the uses/methods described above: (i) enhance solubilisation of the Agent in an aqueous medium with a pH value similar to those found in the upper GI tract of a human (for example pH 4.5 to 8); and (ii) inhibit precipitation of the Agent from an aqueous solution upon an increase in pH of the aqueous solution, particularly when the pH of the aqueous solution is increased from a value similar to the gastric pH in a human to a pH similar to that in the upper intestine in a human.

Suitable water-soluble cellulose ethers or esters thereof and water-soluble acids for use in the above uses and methods of the invention are as hereinbefore defined in relation to any of the pharmaceutical compositions described herein.

The invention is illustrated below by the following non-limiting examples, wherein unless stated otherwise, the Agent is the free base form of formula I.

In the Examples the following abbreviations have been used:

HPLC: High performance liquid chromatography;

ACN: Acetonitrile;

HPMC: Hydroxypropyl methylcellulose;

PVA: Polyvinylalcohol;

PVP: Polyvinylpyrrolidone;

MC: Methylcellulose;

HEC: Hydroxyethylcellulose;

HPC: Hydroxypropylcellulose; and

HPMC AS: Hydroxypropyl methylcellulose acetate succinate.

In the examples all parts and % quoted are by weight unless specified otherwise

### BRIEF DESCRIPTION OF FIGURES

FIG. 1 shows the effectiveness of a composition according to the present invention in inhibiting precipitation of the Agent when the pH of the medium in which the composition is dissolved is increased from 1.5 to 6.5 in the pH Shift Dissolution test described in the Examples. In FIG. 1 the diamond data points represent the composition of Example 5 according to the present invention (Agent, HPMC and fumaric acid). The triangular data points represent Comparative Example 1 containing the Agent and fumaric acid (no HPMC present). The circular points represent Comparative Example 2 containing the Agent and HPMC (no fumaric acid present) and the square Data points represent a Control sample of the Agent alone (no fumaric acid or HPMC present).

FIG. 2 illustrates the improved solubilisation in a pH 6.5 dissolution medium of a composition according the present invention (Example 5) compared to other Comparative formulations containing the Agent. In FIG. 2, the data points shown as “+” represent dissolution of the composition according to the Invention (Example 5: Agent, HPMC and fumaric acid). The triangles represent Comparative Example 1 containing the Agent and fumaric acid. The diamond data points represent Comparative Example 2 containing the Agent and HPMC. The data points shown as an “×” represent dissolution of the fumarate salt of the Agent (Comparative Example 4). The square data points represent a control sample of the Agent alone. The circles represent a Comparative Example in which a composition containing the Agent and HPMC is added to the dissolution medium containing pre-dissolved fumaric acid.

FIG. 3 shows the dissolution at pH 6.5 of various immediate release formulations of the Agent. The data points shown as an “×” represent the solid dispersion formulation of Example 15. The solid circles represent a dry physical mix formulation of Example 5. The triangles represent data from the wet granulate formulation of Example 14. By way of comparison, FIG. 3 also includes data from the Control sample of Agent alone (diamond data points) and a comparative film coated tablet formulation (Comparative Example 8) which contains HPMC but no water-soluble acid (square data points).

FIG. 4 shows the in-vivo plasma concentration of the Agent following administration of various compositions containing the Agent to a group of 6 fasted male beagle dogs. FIG. 4a shows the variable and sometimes low plasma concentrations obtained following administration of the comparative composition of Comparative Example 7. In contrast FIGS. 4b and 4c illustrate the less variable plasma concentrations obtained following administration of Examples 13 and 14 respectively to the same dogs.

Similarly FIG. 5 shows the less variable in-vivo plasma levels in a group of 6 fasted male beagle dogs following administration of the solid dispersion formulation of Example 15 according to the invention composition (FIG. 5b) compared to the highly variable plasma concentrations observed when the composition of comparative Example 7 was administered to the same group of dogs (FIG. 5a).

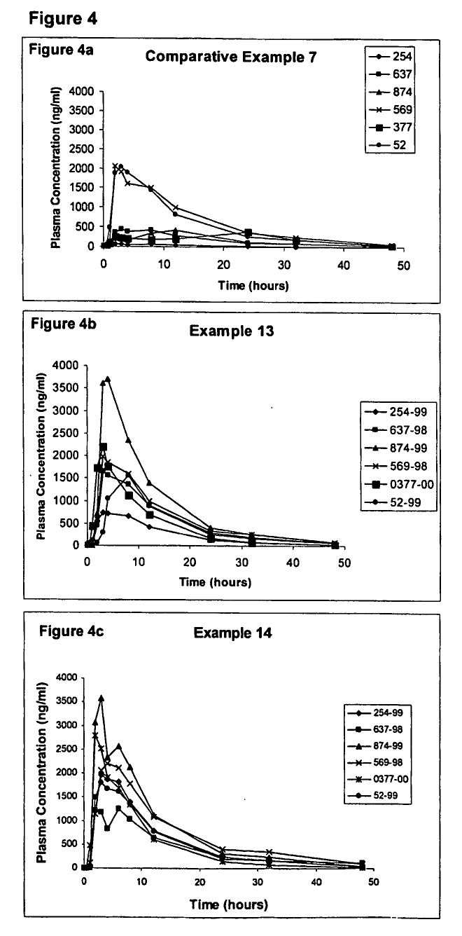

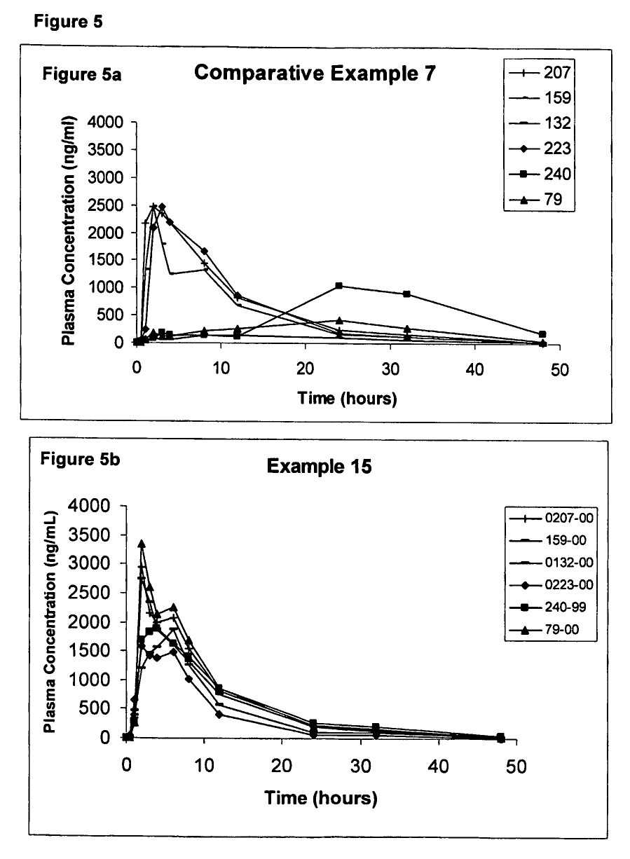

In FIG. 5 the same shaped data points are used in FIGS. 5a and 5b to represent the plasma levels in an individual dog used in the experiment. Thus the relative plasma levels in an individual dog following administration of a particular composition can be compared by comparing the relevant data set in FIG. 5a with the same shaped data points in FIG. 5b. The same convention is used in FIGS. 4a, 4b and 4c.

### EXAMPLES 1 TO 12

### Dry Physical Mixture Compositions

The compositions of Examples 1 to 12 shown in Table 1 were prepared by mixing a stock composition of the Agent and water-soluble cellulose in the weight ratio shown in Table 1. The stock composition was mixed in a 70 ml mortar with a pestle until a homogeneous composition was obtained (typically after about 5 minutes mixing). A second stock composition of the Agentiwater-soluble cellulose physical mix and the acid in the weight ratios of Agent:cellulose:acid required to give the composition shown in Table 1 was subsequently prepared. The second stock composition was mixed in a 70 ml mortar with a pestle until a homogeneous composition was obtained (typically after about 2.5 minutes mixing). The required total weight of the composition (Agent+cellulose+acid) shown in Table 1 was taken from the Agent/water-soluble cellulose/acid stock composition to give the specific Example composition shown in Table 1.

TABLE 1

| | | Cellulose Ether or | |
|---|---|---|---|
| | Agent | Ester of Cellulose | Water-soluble Acid |
| Example | (mg) | Ether (mg) | (mg) |
| 1 | 250 | HPMC (75 mg) | Malic acid (350 mg) |
| 2 | 250 | HPMC (75 mg) | Maleic acid (350 mg) |
| 3 | 250 | HPMC (75 mg) | Succinic acid (350 mg) |
| 4 | 250 | HPMC (75 mg) | Tartaric acid (350 mg) |
| 5 | 250 | HPMC (75 mg) | Fumaric acid (325 mg) |
| 6 | 250 | HPMC (75 mg) | Sulfamic acid (350 mg) |
| 7 | 250 | HPMC (75 mg) | Citric acid (353 mg) |
| 8 | 250 | HPMC (75 mg) | Benzoic acid (350 mg) |
| 9 | 250 | HPMC (75 mg) | Glutamic acid (350 mg) |
| 10 | 250 | HPMC (75 mg) | Ascorbic acid (350 mg) |
| 11 | 250 | HPMC (75 mg) | Sorbic acid (350 mg) |
| 12 | 250 | HPMC acetate | Fumaric acid (325 mg) |
| | | succinate (75 mg) | |

### EXAMPLE 13

The composition shown below was prepared as a dry physical mixture. The drug and excipients were weighed into a mortar and mixed with a pestle until a homogenous composition was obtained.
Agent 250 mg
Fumaric Acid 350 mg
Hydroxypropyl methylcellulose 195 mg
D-Mannitol 100 mg
Crospovidone 100 mg
Sodium lauryl sulfate 5 mg

### EXAMPLE 14

### Wet Granulate Formulation

The composition shown below was prepared as a wet granulate formulation using the following method:
Agent 250 mg
Fumaric acid 350 mg
HPMC 400 mg
D-Mannitol 340 mg
Crospovidone 35 mg
Sodium lauryl sulfate 13.9 mg

A “base granule composition” (10 g) was prepared by mixing the Agent (2.5 g), HPMC (4 g), and fumaric acid (3.5 g) in a mortar with a pestle until a visually homogeneous composition was obtained. Water (approximately 0.7 g) was added to the composition and mixed with a pestle to give a wet mass of material. This was then sieved through a 1 mm sieve and dried for more than 2 hours under vacuum at 40° C. to give the “base granule composition”

A “bulk excipient composition” comprising D-mannitol (2.45 g), crospovidone (0.25 g) and sodium lauryl sulfate (0.10 g) was prepared by mixing in a mortar using a pestle to give a homogenous composition.

1 g of the “base granule” formulation was mixed with 0.39 g of the “bulk excipient composition”. The resulting mixture was added to a capsule which was tumble mixed for more than 2 hours to give the composition of Example 14.

### EXAMPLE 15

### Solid Dispersion Formulation

The composition shown below was prepared as a solid dispersion composition using the method described below:
Agent 250 mg
HPMC 156 mg
Fumaric acid 219 mg
Lactose 141 mg
Sodium lauryl sulphate 16 mg

HPMC (1.5 g) and Agent (2.4 g) were dissolved in a 50/50 (v/v) methanol/dichloromethane mixture (130 ml). Fumaric acid (2.1 g) was dissolved in methanol (35 ml) with slight warming and added to the Agent HPMC solution, together with water (35 ml). The resulting solution was then spray dried and the solid dispersion composition collected.

A bulk excipient composition was prepared by mixing the lactose and sodium lauryl sulphate in the required weight ratio of lactose: sodium lauryl sulfate in a mortar. The components were mixed with a pestle until a visually homogeneous composition was obtained.

The required weight of the bulk excipient composition and the spray dried solid dispersion composition were added to a capsule and the compositions tumble mixed for 20 minutes to give the composition of Example 15.

### EXAMPLES 16 To 22

### Dry Physical Mixture Comupositions

The compositions of Examples 16 to 22 shown in Table 1a were prepared by mixing a stock composition of the Agent and water-soluble cellulose in the weight ratio shown in Table 1a. The stock composition was mixed in a 70 ml mortar with a pestle for about 5 minutes. A second stock composition of the Agent/water-soluble cellulose physical mix and the acid in the weight ratios of Agent:cellulose:acid required to give the composition shown in Table 1a was subsequently prepared. This stock composition was mixed in a 70 ml mortar with a pestle for about 2.5 minutes. The required total weight of the composition (Agent+cellulose+acid) shown in Table 1a was taken from the Agent/water-soluble cellulose/acid stock composition to give the specific Example composition shown.

A second batch of Example 16 was prepared by mixing a stock composition of the Agent, water-soluble cellulose and acid in the weight ratio shown in Table 1a. The stock composition was mixed in a mortar with a pestle until a homogenous mixture was obtained. This batch was tested in the pH6.5 test (Table 4).

The specific composition of each of Examples 16 to 22 was weighed into a vial.

TABLE 1a

| | | Cellulose Ether or | |
|---|---|---|---|
| | Agent | Ester of Cellulose | Water-soluble Acid |
| Example | (mg) | Ether (mg) | (mg) |
| 16 | 250 | MC (75 mg) | Fumaric acid (325 mg) |
| 17 | 250 | MC (75 mg) | Malic acid (350 mg) |
| 18 | 250 | HEC (75 mg) | Fumaric acid (325 mg) |
| 19 | 250 | HPC (75 mg) | Fumaric acid (325 mg) |
| 20 | 250 | HEC (75 mg) | Malic acid (350 mg) |
| 21 | 250 | HPC (75 mg) | Malic acid (350 mg) |
| 22 | 250 | HPMC AS (75 mg) | Malic acid (350 mg) |

### COMPARATIVE EXAMPLE 1

### Dry Physical Mixture (No Water-Soluble Cellulose Ether)

The following dry physical mixture was prepared by mixing a stock composition of the Agent and acid in the weight ratio shown below. The stock composition was mixed in a mortar with a pestle until a homogenous mixture was obtained:
Agent 250 mg
Fumaric Acid 325 mg.

### COMPARATIVE EXAMPLE 2

### Dry Physical Mixture (No Water-Soluble Acid)

The following dry physical mixture was prepared by mixing a stock composition of the Agent and water-soluble cellulose in the weight ratio shown below. The stock composition was mixed in a mortar with a pestle until a homogenous mixture was obtained:
Agent 250 mg
HPMC 75 mg

COMPARATIVE EXAMPLE 3

### Water-Soluble Acid Dissolved in Dissolution Medium

The following composition was prepared as described below:
Agent 250 mg
HPMC 75 mg
Fumaric Acid 325 mg (pre-dissolved in dissolution medium)
   In Comparative Example 3, fumaric acid (325 mg) was pre-dissolved in dissolution medium (pH 6.5) comprising of 0.07N HCl, sodium chloride (0.2% w/v) and 10 ml of a 2.5M KH₂PO₄/16.72% (w/v) NaOH solution (prepared as described below in relation to the pH shift dissolution test). The Agent and HPMC were prepared as a dry physical mixture using an analogous method to that described in Comparative Example 2. This composition was then added to the dissolution medium.

### COMPARATIVE EXAMPLE 4

### Fumarate Salt of the Agent

The Agent (1 g) was placed into a 250 ml conical flask. Methanol (100 ml) was added to the flask and heated on a steam bath until the Agent had completely dissolved. Fumaric acid (390 mg) was weighed directly into a glass vial and dissolved in 15 ml of Methanol. The solution was warmed on the steam bath and then added to the hot Agent solution and swirled to form a homogeneous solution. The resulting solution was allowed to cool. The crystals formed on cooling were isolated by filtration and dried overnight in a vacuum oven.

347.5 mg of the fumarate salt of the Agent was used in the pH 6.5 dissolution test described below.

### COMPARATIVE EXAMPLE 5

### Water-Soluble Cellulose Ether Replaced by Polyvinylpyrrolidone

The following dry physical mixture was prepared by mixing a stock composition of Agent, polymer and acid in the weight ratios of Agent:polymer:acid required to give the composition shown below. The stock composition was mixed in a mortar and pestle until a homogeneous composition was obtained. The required total weight of the composition (Agent+polymer+acid) shown below was taken from the stock composition to give the specific Example composition shown:
Agent 250 mg
Polyvinylpyrrolidone K25 75 mg
Fumaric Acid 325 mg

### COMPARATIVE EXAMPLE 6

### Water-Soluble Cellulose Ether Replaced by Polyvinyl Alcohol

The following dry physical mixture was prepared using an analogous method to that described for the preparation of Comparative Example 5:
Agent 250 mg
Polyvinyl Alcohol 75 mg
Fumaric Acid 325 mg

### COMPARATIVE EXAMPLE 7

### Coated Tablet Formulation (No Water-Soluble Acid)

Tablet core

| | |
|---|---|
| The Agent | 250.0 mg |
| Lactose monohydrate | 163.5 mg |
| Microcrystalline cellulose | 50.0 mg |
| Croscarmellose sodium | 20.0 mg |
| Povidone | 10.0 mg |
| Sodium lauryl sulphate | 1.5 mg |
| Magnesium stearate | 5.0 mg |

Tablet coating

| | |
|---|---|
| Hydroxypropyl methylcellulose | 7.65 mg |
| Polyethylene glycol 300 | 1.5 mg |
| Titanium Dioxide | 0.50 mg |
| Yellow ferric oxide | 0.90 mg |
| Red ferric oxide | 0.90 mg |

The above formulation was prepared by conventional wet granulation, compression and film coating processes. The Agent, lactose monohydrate, microcrystalline cellulose and croscarmellose sodium were mixed together in a high shear granulator to produce a homogeneous mix. An aqueous solution of the povidone and sodium lauryl sulphate was then added to the powders with mixing until a suitable wet mass was obtained. The wet granules were passed through a suitable screen to remove large particles, then dried. The dried granules were then passed through a further screen and blended with pre-milled magnesium stearate. The resultant granules were compressed into tablet cores, which were then coated using a conventional pan coater. The film coat was applied by spraying an aqueous suspension of the hydroxypropyl methylcellulose, polyethylene glycol 300, talc, titanium dioxide and yellow ferric oxide onto the tablet cores. The yellow iron oxide, titanium dioxide, talc and a portion of the HPMC606 in the film coat were provided in Opaspray Yellow M-1-22842, ex. Colorcon Ltd, Dartford, Kent, UK.

### COMPARATIVE EXAMPLE 8

### Coated Tablet Formulation

Tablet core

| | |
|---|---|
| The Agent | 250.0 mg |
| Lactose monohydrate | 163.5 mg |
| Microcrystalline cellulose | 50.0 mg |
| Croscarmellose sodium | 20.0 mg |
| Povidone | 10.0 mg |
| Sodium lauryl sulphate | 1.5 mg |
| Magnesium stearate | 5.0 mg |

Tablet coating

| | |
|---|---|
| Hydroxypropyl methylcellulose | 8.16 mg |
| Polyethylene glycol 300 | 1.60 mg |
| Talc | 1.18 mg |
| Titanium Dioxide | 1.18 mg |
| Yellow ferric oxide | 0.04 mg |

The above formulation was prepared using an analogous method to that described for the preparation of Comparative Example 7. The film coating comprising the red and yellow iron oxides, the titanium dioxide and a portion of the HPMC 606 were provided in Opaspray Brown M-1-25092, Colorcon Ltd, Dartford, Kent, UK. This concentrate and titanium dioxide (Opaspray™ Brown M-1-25092, ex Colorcon) was diluted in a base containing water, polyethylene glycol 300 and HMPC to provide the film coat which was then applied to the tablet cores in a pan coater.

### COMPARATIVE EXAMPLES 9 TO 12

The following comparative dry physical mixture were prepared using an analogous method to that described for the preparation of Examples 16 to 22 above, but in the absence of the acid to give the Comparative Examples shown in Table 1b:

TABLE 1b

| | | Cellulose Ether or | |
|---|---|---|---|
| Comparative | Agent | Ester of Cellulose | Water-soluble |
| Example | (mg) | Ether (mg) | Acid (mg) |
| Comparative 9 | 250 | HPMC AS (75 mg) | — |
| Comparative 10 | 250 | MC (75 mg) | — |
| Comparative 11 | 250 | HEC (75 mg) | — |
| Comparative 12 | 250 | HPC (75 mg) | — |

Control
Agent 250 mg (No water-soluble acid or cellulose ether/ester thereof)

The excipients used in the above Examples and Comparative Examples are all commercially available and had the following characteristics:
HPMC was Pharmacoat 606, ex. Shin-Etsu, viscosity 6 cP (2 w/v % aqueous solution at 20° C. as measured by USP method USP 24, NF 19, 2000, p 843-844 and USP 24, NF 19, 2000 p 2002-2003).
HPMC acetate succinate was Aqoat AS-LG ex. Shin-Etsu. Kinematic viscosity 2.4-3.6 mm²/s (measured using the method described in Japanese Pharmaceutical Excipients 1993).
Polyvinylpyrrolidone was Kollidon 25 ex. BASF, K value 22.5-26.7, approximate molecular weight 30 000, dynamic viscosity 3.5-5.5 mPas (10% w/v aqueous solution at 20° C.) as described in Handbook of Pharmaceutical Excipients, 3^{rd} edition, 2000, American Pharmaceutical Association and the Pharmaceutical Press.
Polyvinyl alcohol was Airvol 205 ex. Air Products and Chemicals Inc.
HEC ex. Aldrich, viscosity spec. 80-125 cP (2% w/v solution in water at 25° C.). Actual viscosity 107 cP. Mw approximately 250 000.
HPC was HPC LH-21, ex. Shin-Etsu.
MC was Methocel MC ex. Fluka, viscosity spec. 10-25 cP (2% w/v solution in water at 20° C.).
   pH Shift Dissolution Test

This test simulates the change in in-vivo pH experienced upon moving from the stomach to the upper-intestine and measures the rate of precipitation of Agent from solution upon increase in pH.

The Composition to be tested was weighed into a vial and added to 450 ml of dissolution medium comprising 0.07N HCl (approximately pH 1.5) and sodium chloride (0.2% w/v). The vial was then rinsed with a further 50 ml of fresh dissolution medium and the washings added to the main dissolution medium to ensure complete transfer of the composition (total volume of medium 500 ml). The Control sample was weighed into a volumetric flask and pre-dissolved in 50 ml of dissolution medium via sonication. The composition was then added to 400 ml of dissolution medium. The flask was rinsed with a further 50 ml of fresh dissolution medium and the washings added to the main dissolution medium to ensure complete transfer of the composition (total volume of medium=500 ml). After 60 minutes at 37° C. (paddle speed 100 rpm) a sample (5 ml) was taken and 5 ml of fresh medium was added to retain a constant volume of dissolution medium. HPLC analysis (as described below) of this sample confirmed that 100% of the Agent was in solution.

A 2.5M KH₂PO₄/16.72% (w/v) NaOH buffered solution was prepared by dissolving KH₂PO₄ (34.02 g) and NaOH (16.72 g) in MilliQ water (about 75 ml). The resulting solution was then made up to 100 ml with MilliQ water.

A portion of the 2.5M KH₂PO₄/16.72% (w/v) NaOH solution (10 ml) was then added to the dissolution medium to shift the pH from 1.5 to 6.5. Samples (5 ml) were then removed with a plastic syringe at 2, 5, 15, 30, 45 and 60 minutes after pH adjustment and fresh medium (pH 6.5) added to maintain a constant volume of dissolution medium. Each sample was centrifuged (14,000 rpm) at ambient temperature for 15 minutes and then analysed by HPLC to give the concentration of the Agent in the sample using the following conditions:
Eluent: 38% ACN/62% water/0.6% ammonium acetate
column: 10 cm×3 mm (internal diameter) INERTSIL ODS-3¹. (with guard)
detection wavelength: 247 nm
flow rate: 0.9 ml/min
injection volume: 20 μl
retention time: approximately 6 minutes

Footnote [1]: column ex Hichrom containing 3 μm beads.

pH Shift Dissolution Results

Table 2 shows the results from the pH shift dissolution test for Example 1, 5, 12, 16 to 22 together with the results from the Control sample of the Agent alone and from Comparative Examples 1, 2, 4, 9 to 12. In Table 2, the third column indicates the % by weight of the Agent that has dissolved in the acidic dissolution medium after 60 minutes (those cases that indicate >100% result from the experimental error associated with the HPLC analysis). Columns 4, 5 and 6 show the % Agent in solution at 2 minutes, 30 and 60 minutes following adjustment of the dissolution medium from pH 1.5 to pH 6.5. The mean pH of the dissolution medium 60 minutes after pH adjustment from pH 1.5 to pH 6.5 is shown in the final column of Table 2.

TABLE 2

| | | % Agent in Solution | | | | Mean pH of medium |
|---|---|---|---|---|---|---|
| | | t = 0 | | | | at 60 minutes after |
| Example | Composition | (˜pH 1.5) | t = 2 mins | t = 30 mins | t = 60 mins | pH adjustment (n = 2) |
| 1 | Agent, Malic Acid, HPMC | 93.87 | 93.91 | 94.88 | 91.87 | 6.01 |
| 5 | Agent, Fumaric Acid, HPMC | 93.53 | 93.87 | 93.87 | 93.38 | 5.94 |
| 12 | Agent, Fumaric Acid, HPMC AS | 98.18 | 79.53 | 65.22 | 67.09 | 5.94 |
| 16 | Agent, Fumaric Acid, MC | 100.72 | 100.83 | 101.47 | 105.22 | 5.96 |
| 17 | Agent, Malic Acid, MC | 99.59 | 98.92 | 101.12 | 102.51 | 5.98 |
| 18 | Agent, Fumaric Acid, HEC | 100.58 | 100.03 | 99.43 | 57.36 | 5.93 |
| 19 | Agent, Fumaric Acid, HPC | 100.79 | 98.69 | 95.47 | 42.72 | 5.91 |
| 20 | Agent, Malic Acid, HEC | 98.20 | 97.02 | 88.94 | 27.33 | 5.95 |
| 21 | Agent, Malic Acid, HPC | 100.67 | 97.58 | 88.47 | 28.14 | 5.94 |
| 22 | Agent, Malic Acid, HPMC AS | 97.30 | 73.91 | 63.04 | 65.19 | 5.96 |
| Comparative 1 | Agent, Fumaric Acid | 97.53 | 89.49 | 38.48 | 9.20 | 5.92 |
| Comparative 2 | Agent, HPMC | 90.00 | 85.62 | 21.24 | 12.36 | 6.42 |
| Comparative 4 | Fumarate Salt | 97.74 | 19.56 | 5.16 | 3.30 | 6.32 |
| Comparative 9 | Agent, HPMC AS | 103.91 | 55.88 | 43.55 | 37.62 | 6.42 |
| Comparative 10 | Agent, MC | 101.93 | 73.07 | 36.63 | 16.52 | 6.43 |
| Comparative 11 | Agent, HEC | 103.22 | 66.71 | 10.72 | 9.28 | 6.41 |
| Comparative 12 | Agent, HPC | 102.53 | 61.86 | 6.92 | 6.68 | 6.40 |
| Control | Agent alone | 102.61 | 6.50 | 3.04 | 2.27 | 6.55 |

Table 2 clearly shows that the compositions containing the Agent, water-soluble cellulose ether or ester of water-soluble cellulose ether and acid (Examples 1, 5, 12, 16 to 22) retain high levels of the Agent in solution. This is in marked contrast to the Comparative Examples and the Control sample of the Agent alone. For all of these compositions the Agent rapidly precipitated from solution upon shift of the pH to 6.5.

Example 5 (a dry physical mixture according to the present invention) retains substantially all of the Agent in solution (93.38%) 60 minutes after shifting the pH of the medium from 1.5 to pH 6.5 (compare column 3 (% Agent in solution just prior to increasing pH) with columns 4, 5 and 6 (% Agent in solution at 2, 30 and 60 minutes following pH shift). In contrast just 2.27% of the Agent is in solution when the Control sample of the Agent alone was used.

Table 2 also shows that the compositions of Comparative Examples 1 and 2 which contain the Agent and fumaric acid, or the Agent and HPMC, have only a relatively small effect upon inhibiting precipitation of the Agent compared to the Control sample of the Agent alone. Similarly, Comparative Example 4 (fumarate salt of the Agent) does not significantly inhibit the precipitation of the Agent from solution upon pH increase.

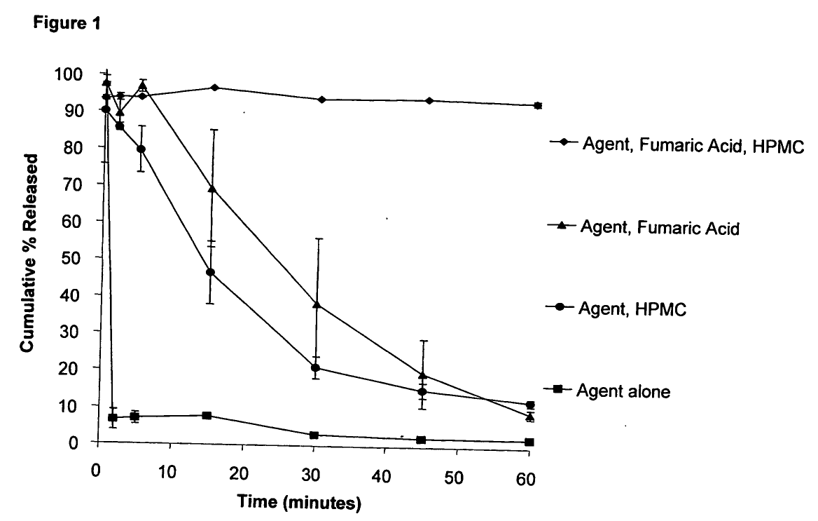

FIG. 1 illustrates the marked effect upon reduction of precipitation of the Agent of Example 5 compared to any of Comparative Examples 1 and 2 or the Control sample of the Agent alone in the pH shift dissolution test.

FIG. 1 clearly illustrates the synergy obtained by the compositions according to the present invention comprising the Agent, a water-soluble acid and a water-soluble cellulose ether.

pH 6.5 Dissolution Method

The following test measures dissolution of a composition in a pH 6.5 dissolution medium and simulates the pH environment found in the upper intestine.

The composition to be tested was placed into a 500 ml of a stirred dissolution medium at pH 6.5 at 37° C. (paddle speed 100 rpm). The dissolution medium comprised 0.07N HCl, sodium chloride (0.2% w/v), and 10 ml of a 2.5M KH₂PO₄/16.72% (w/v) NaOH solution (the KH₂PO₄ buffer solution was prepared as described above). Samples (5 ml) of dissolution medium were removed with a plastic syringe at 5, 10, 20 and 30 (and for some examples 45 and 60 minutes) after addition of the composition to the dissolution medium. A fresh sample of dissolution medium was added after every sampling time point to maintain a constant volume of 500 ml dissolution medium throughout the test.

Each sample was centrifuged (14,000 rpm) at ambient temperature for 15 minutes and then analysed by HPLC, using the conditions described in the pH shift test, to determine the concentration of the Agent in the sample.

Notes:

The dry physical mixture compositions in the Examples and Comparative Examples and the Control sample described above were added to the dissolution medium by placing the required weight of composition to be tested in a vial and pouring the composition into 450 ml of the dissolution medium. The vial was then rinsed with a further 50 ml of fresh dissolution medium and the washings added to the main dissolution medium to ensure complete transfer of the composition (total volume of dissolution medium=500 ml).

In Example 14 (wet granulation formulation) and Example 15 (solid dispersion formulation), the composition to be tested was weighed into gelatin capsules and added to 500 ml of dissolution medium at 37° C.

Comparative Example 8 was added directly to 500 ml of the dissolution medium at 37° C.

Results: pH 6.5 Dissolution

Table 3 shows the results from the pH 6.5 dissolution test for the composition of Example 5 according to the present invention, together with the results from the Control sample of the Agent alone and Comparative Examples 1 to 4. In Table 3, the fourth column indicates the % by weight of the Agent that has dissolved in the dissolution medium 60 minutes after immersion of the composition into the dissolution medium. The pH of the dissolution medium at 60 minutes is shown in the last column of Table 3.

TABLE 3

| | | | | Mean pH of |
|---|---|---|---|---|
| | | % Agent in solution | % Agent in solution | medium at |
| | | after 30 minutes | after 60 minutes | 60 minutes |
| Example | Composition | in pH 6.5 medium | in pH 6.5 medium | (n = 2) |
| 5 | Agent, HPMC, Fumaric Acid | 68.37 | 52.86 | 6.0 |
| Comparative 1 | Agent, Fumaric Acid | 4.91 | 4.75 | 6.13 |
| Comparative 2 | Agent, HPMC | 1.97 | 2.04 | 6.5 |
| Comparative 3 | Agent, HPMC (Fumaric Acid in medium) | 7.13 | 7.24 | 5.94 |
| Comparative 4 | Fumarate Salt | 1.54 | 1.21 | 6.33 |
| Control | Agent alone | 1.52 | 1.40 | 6.52 |

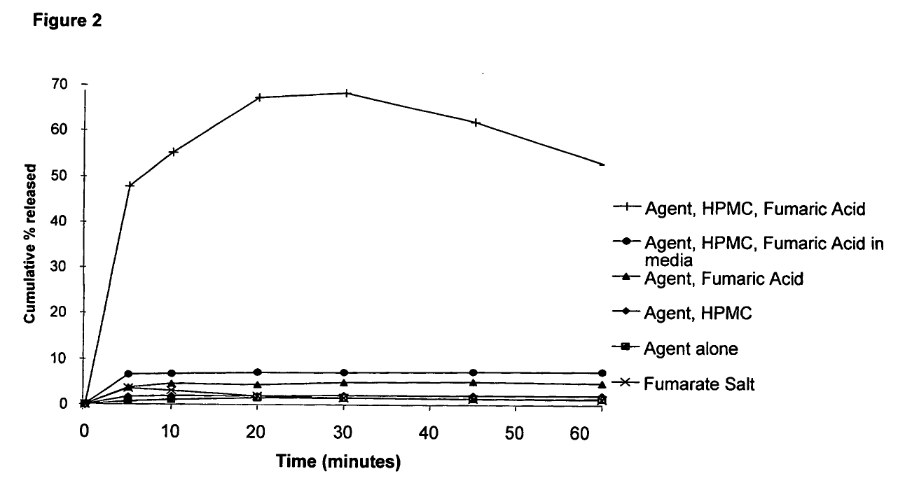

FIG. 2 illustrates the pH 6.5 dissolution of the compositions shown in Table 3. Table 3 and FIG. 2 show the marked solubilisation of the Agent in the composition of Example 5. The solubilisation of the Agent is significantly increased at 60 minutes in pH 6.5 medium when both the HPMC and fumaric acid are present in the formulation compared to the Agent alone (Control sample) or the Agent in combination with fumaric acid (Comparative 1) or the Agent and HPMC (Comparative 2). In the composition of Example 5 after 60 minutes in the pH 6.5 medium approximately 53% of the Agent was in solution. In comparison only 1.40% of the Agent was in solution for the Control sample of the Agent alone and just 4.75% and 2.04% for Comparative Examples 1 and 2 respectively.

Pre-dissolving the fumaric acid in the dissolution medium and adding the Agent and HPMC (Comparative Example 3) did not significantly solubilise the Agent with only 7.24% of the Agent in solution after 60 minutes. This suggests the fumaric acid must be in intimate contact with the Agent and HPMC in order to enhance the solubilisation of the Agent.

The fumarate salt of the Agent alone used in Comparative Example 4 also did not significantly solubilise the Agent with just 1.21% in solution after 60 minutes.

The pH of the dissolution medium 60 minutes after addition of Example 5, a composition according to the invention which contained fumaric acid, did not fall below pH 6.0 indicating the enhanced solubilisation of the Agent demonstrated by Example 5 is not merely the result of the acid lowering the bulk pH of the dissolution medium.

Effect of the Polymer Type on Dissolution at pH 6.5

Table 4 shows the effect of different water-soluble cellulose ethers/esters thereof on dissolution of the Agent in pH 6.5 media.

TABLE 4

| | | | | Mean pH of |
|---|---|---|---|---|
| | | % Agent in solution | % Agent in solution | medium at end |
| | | after 5 minutes | after 30 minutes | of experiment |
| Example | Composition | in pH 6.5 medium | in pH 6.5 medium | (n = 2) |
| 5 | Agent, HPMC, Fumaric Acid | 47.87 | 68.37 | 6.0 |
| 12 | Agent, HPMCAS, Fumaric Acid | 25.05 | 24.61 | 6.13 |
| 16 | Agent, Fumaric Acid, MC | 60.50 | 40.92 | 6.18 |
| 18 | Agent, HEC, Fumaric Acid | 17.45 | 21.06 | 5.87 |
| 19 | Agent, HPC, Fumaric Acid | 17.48 | 17.13 | 5.88 |
| Comparative 5 | Agent, PVP, Fumaric Acid | 6.67 | 8.71 | 5.9 |
| Comparative 6 | Agent, PVA, Fumaric Acid | 6.15 | 6.80 | 6.1 |
| Comparative 1 | Agent, Fumaric Acid | 3.77 | 4.91 | 6.13 |
| Comparative 2 | Agent, HPMC | 1.65 | 1.97 | 6.5 |
| Control | Agent alone | 0.70 | 1.52 | 6.52 |

Table 4 shows that the compositions according to the present invention containing a water-soluble cellulose ether or ester thereof exhibit significantly higher solubilisation at pH 6.5 compared to Comparative compositions containing a different water-soluble polymer. In Table 4, the third and fourth columns indicate the % by weight of the Agent that has dissolved in the pH 6.5 dissolution medium after 5 minutes and 30 minutes respectively. The pH of the dissolution medium at the end of the experiment is shown in column 5 of Table 4. In Example 12 and Comparative Examples 5 and 6 the final pH of the media was measured after 30 minutes. In Examples 5, 16, 18, 19, Comparative Examples 1 and 2 and the Control, the final pH of the medium was measured after 60 minutes.

Table 4 shows that for the composition of Example 5, after 30 minutes in the pH 6.5 medium approximately 68% of the Agent was in solution. In comparison only 8.71% and 6.80% of the Agent was in solution for Comparative Examples 5 and 6 respectively, and just 1.52% of the Agent was in solution for the Control sample of the Agent alone at the same 30 minute time point. Examples 12, 16, 18, 19, in which the HPMC in Example 5 has been substituted for an alternative water-soluble cellulose ether or ester of water-soluble cellulose ether, continue to exhibit significantly enhanced solubilisation at pH6.5 compared to the Comparative Examples and the Control sample of the Agent alone.

Effect of Acid upon Dissolution in pH 6.5 Dissolution Medium

Table 5 shows the results from the pH 6.5 dissolution test for Examples 1 to 11 described above which contained the Agent (250 mg), HPMC (75 mg) and the acid as shown in the second column of Table 5. In each case 350 mg of acid was used except in Examples 5 and 7 which contained 325 mg and 353 mg respectively. Table 5 also shows the data from the Control sample of the Agent alone and data from Comparative Examples 1 to 4. In Table 5, the third and fourth columns indicate the % by weight of the Agent that has dissolved in the pH 6.5 dissolution medium after 5 minutes and 30 minutes respectively.

TABLE 5

| | | | | Mean pH of |
|---|---|---|---|---|
| | | % Agent in solution | % Agent in solution | medium at end |
| | | after 5 minutes | after 30 minutes | of experiment |
| Example | Composition | in pH 6.5 medium | in pH 6.5 medium | (n = 2) |
| 1 | Agent, HPMC, Malic Acid | 88.28 | 93.07 | 5.89 |
| 2 | Agent, HPMC, Maleic Acid | 86.26 | 90.68 | 6.0 |
| 3 | Agent, HPMC, Succinic Acid | 82.32 | 85.53 | 5.95 |
| 4 | Agent, HPMC, Tartaric Acid | 70.16 | 70.93 | 5.90 |
| 5 | Agent, HPMC, Fumaric Acid | 47.87 | 68.37 | 6.0 |
| 6 | Agent, HPMC, Sulfamic Acid | 86.48 | 65.39 | 6.05 |
| 7 | Agent, HPMC, Citric Acid | 67.42 | 58.42 | 6.06 |
| 8 | Agent, HPMC, Benzoic Acid | 17.90 | 35.57 | 6.28 |
| 9 | Agent, HPMC, Glutamic Acid | 31.56 | 18.89 | 6.20 |
| 10 | Agent, HPMC, Ascorbic Acid | 48.76 | 16.80 | 6.19 |
| 11 | Agent, HPMC, Sorbic Acid | 12.22 | 14.72 | 6.26 |
| Comparative 1 | Agent, Fumaric Acid | 3.77 | 4.91 | 6.13 |
| Comparative 2 | Agent, HPMC | 1.65 | 1.97 | 6.5 |
| Comparative 3 | Agent, HPMC (Fumaric Acid in medium) | 6.50 | 7.13 | 5.94 |
| Comparative 4 | Fumarate Salt | 3.52 | 1.54 | 6.33 |
| Control | Agent alone | 0.70 | 1.52 | 6.52 |

Table 5 clearly shows that the compositions of Examples 1 to 11 according to the invention solubilise the Agent in pH 6.5 medium to a greater extent than the Control sample, containing the Agent alone, and Comparative Examples 1 to 4. In Example 1 (containing the Agent, HPMC and malic acid) 93.07% of the Agent was in solution after 30 minutes in pH 6.5 medium compared to just 1.52% of the Control sample. Table 5 also shows that the compositions of Comparative Examples 1 to 4 have relatively low dissolution compared to the composition according to the invention. The pH of the dissolution medium at the end of the experiment is shown in the fifth column of Table 5. For Examples 1 to 4, 6, 8 to 11 the pH was measured after 30 minutes. In Examples 5 and 7, Comparative Examples 1 to 4 and the Control, the pH of the medium at the end of the experiment was measured after 60 minutes.

Effect of Formulation Type on pH 6.5 Dissolution

Table 6 shows the results from the pH 6.5 dissolution test for the compositions of Examples 5, 14 and 15 described above according to the invention. By way of comparison Table 6 also shows the data from a Control example of the Agent alone and comparative Example 8, a film coated tablet formulation but which does not contain a water-soluble acid. In Table 6, the third and fourth columns indicate the % by weight of the Agent that has dissolved in the dissolution medium after 5 and 60 minutes respectively. The pH of the dissolution medium at 60 minutes is shown in the last column of Table 6.

TABLE 6

| | | % Agent in solution | % Agent in solution | % Agent in solution | Mean pH of medium |
|---|---|---|---|---|---|
| | | after 5 minutes | after 30 minutes | after 60 minutes | at 60 minutes |
| Example | Formulation type | in pH 6.5 medium | in pH 6.5 medium | in pH 6.5 medium | (n = 2) |
| 5 | Physical Mix | 47.87 | 68.37 | 52.86 | 6.0 |
| 14 | Wet Granulate | 0.0 | 49.72 | 33.61 | 5.94 |
| 15 | Solid dispersion | 0.34 | 41.93 | 59.69 | 6.10 |
| Comparative 8 | Coated Tablet formulation | 2.82 | 2.40 | 2.45 | Not measured |
| | (no water-soluble acid) | | | | |
| Control | Agent alone | 0.70 | 1.52 | 1.40 | 6.52 |

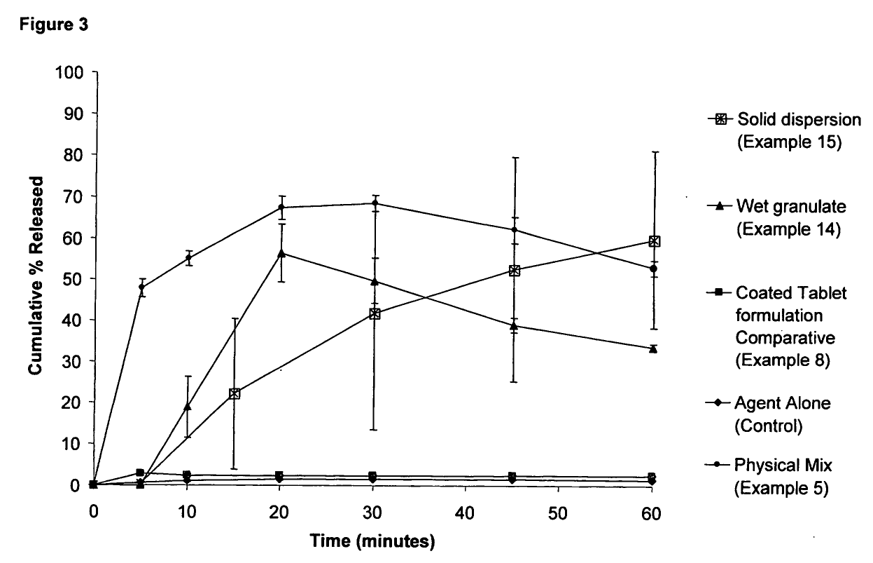

FIG. 3 illustrates the pH 6.5 dissolution of the compositions shown in Table 6. FIG. 3 and Table 6 illustrate that compositions according to the invention prepared as dry physical mixes, wet granulate or solid dispersion formulations all display marked solubilisation of the Agent at 60 minutes compared to the Control example of the Agent alone. In the compositions of Examples 5, 14 and 15 after 60 minutes in the pH 6.5 medium approximately 53%, 34% and 60% of the Agent remained in solution respectively. In comparison only 1.40% of the Agent was in solution for the Control sample of the Agent alone and just 2.45% for Comparative Example 8 (a film coated tablet composition containing HPMC but no water-soluble acid).

In Vivo Administration

The composition of Example 13 according to the invention (physical mixture containing HPMC, fumaric acid, Agent and additional excipients) was orally administered in 5 ml gelatin capsules to one group of 6 fasted male beagle dogs. Following administration of the composition, each dog received 20 mls of water. Whole blood samples (3 ml) were taken from each animal immediately prior to dosing and at pre-determined intervals up to 48 hours post dosing. Blood samples were analysed using High Performance Liquid Chromatography Tandem Mass Spectrometry for plasma concentrations of the Agent.

This procedure above was repeated after a minimum washout period of 1 week in the same group of dogs with the compositions of Example 14 (wet granulation formulation according to the invention) and the composition of Comparative Example 7 (film coated tablet formulation containing HPMC but no water-soluble acid).

FIG. 4 shows the Agent plasma concentrations following administration of the compositions above. FIG. 4a shows the plasma concentrations following administration of the comparative composition of Comparative Example 7. FIG. 4a illustrates that the plasma concentration in the dogs is broadly divided into two groups. Two of the dogs displayed high plasma levels of Agent, however, the other four dogs had much lower plasma levels. There is therefore considerable variability in absorption of the Agent in these dogs, when the Comparative composition containing the Agent was administered.

In contrast FIG. 4b shows that when the composition of Example 13 according to the invention was administered to the same dogs all the dogs showed high plasma concentrations of the Agent and a reduced variability in Agent plasma concentrations. Increased plasma concentrations of the Agent and reduced variability was also observed following administration of the wet granulation formulation of Example 14 according to the invention, as illustrated in FIG. 4c.

The above dosing procedure was repeated using a different group of 6 fasted male beagle dogs, except the plasma concentration of Agent was measured following administration of the solid dispersion composition of Example 15 according to the invention.

For comparison, the same dogs were also given the composition of Comparative Example 7 (film coated tablet formulation containing HPMC but no water-soluble acid).

FIG. 5 shows the results of this experiment with the two compositions. It is clear from FIG. 5b that the composition according to the present invention resulted in high plasma levels in all the dogs with little variability. This is in marked contrast to the plasma levels following administration of the Comparative Example 7 (FIG. 5a), which again shows that only three of the 6 dogs demonstrated high levels of the Agent, whilst the other three dogs only attained low plasma concentrations of the Agent.

These examples clearly show that the compositions according to the present invention give a marked improvement in the extent of in-vivo absorption and systemic exposure of the Agent in those dogs that previously demonstrated low systemic exposure when the Comparative composition was administered. Accordingly, the compositions according to the invention significantly reduced variability of in-vivo plasma levels of the Agent.

1. An immediate release pharmaceutical composition comprising:
(i) 4-(3′-chloro-4′-fluoroanilino)-7-methoxy-6-(3-morpholinopropoxy)quinazoline or a pharmaceutically-acceptable salt thereof (the Agent);
(ii) a water-soluble acid; and
(iii) a water-soluble cellulose ether or an ester of a water-soluble cellulose ether.

2. A pharmaceutical composition according to claim 1 comprising the Agent, a water-soluble acid and a water-soluble cellulose ether.

3. A pharmaceutical composition according to claim 1 comprising the Agent, a water-soluble acid and an ester of a water-soluble cellulose ether.

4. A pharmaceutical composition according to claim 1 or claim 2 comprising the Agent, a water-soluble acid and a water-soluble cellulose ether selected from methyl cellulose, hydroxyethylcellulose, hydroxypropylcellulose and hydroxypropyl methylcellulose.

5. A pharmaceutical composition according to claim 1 or claim 2 comprising the Agent, a water-soluble acid and methyl cellulose.

6. A pharmaceutical composition according to claim 1 or claim 2 comprising the Agent, a water-soluble acid and hydroxypropyl methylcellulose.

7. A pharmaceutical composition according to claim 1 or claim 3 comprising the Agent, a water-soluble acid and an ester of hydroxypropyl methylcellulose or an ester of hydroxypropylcellulose which carries one or more ester groups selected from acetate, succinate, phthalate, isophthalate, terephthalate and trimellitate.

8. A pharmaceutical composition according to claim 1 or claim 2 comprising the Agent, a water-soluble acid and a water-soluble cellulose ether or ester of a water-soluble cellulose ether selected from hydroxypropyl methylcellulose, hydroxypropylcellulose, hydroxyethylcellulose, methylcellulose and hydroxypropyl methylcellulose acetate succinate.

9. A pharmaceutical composition according to any one of the preceding claims wherein the water-soluble acid is solid at ambient temperature.

10. A pharmaceutical composition according to any one of the preceding claims wherein the water-soluble acid is a water-soluble aliphatic mono or poly-carboxylic acid which may be saturated or unsaturated.

11. A pharmaceutical composition according to claim 10 wherein the water-soluble acid is selected from fumaric acid and malic acid.

12. A pharmaceutical composition according to any one of the preceding claims wherein the molar ratio of Agent to acid is from 1:1 to 1:10.

13. A pharmaceutical composition according to any one of the preceding claims wherein the weight ratio of Agent to water-soluble cellulose ether, or ester of water-soluble cellulose ether is from 30:1 to 3:1.

14. A pharmaceutical composition according to claim 1 comprising:
(i) from 10 to 60 parts of the Agent;
(ii) from 2 to 70 parts of a water-soluble cellulose ether selected from methyl cellulose and hydroxypropyl methylcellulose; and
(iii) from 10 to 70 parts of a water-soluble organic acid selected from fumaric acid and malic acid;
wherein all parts are by weight and the sum of the parts (i)+(ii)+(iii)=100;
and wherein the molar ratio of Agent to organic acid is from 1:3 to 1:6.

15. A pharmaceutical composition according to any one of the preceding claims which comprises a physical mixture of the Agent, the water-soluble acid, and the water-soluble cellulose ether and/or ester of a water-soluble cellulose ether.

16. A pharmaceutical composition according to claim 15 which is in the form of an oral immediate release tablet, pellet, granule or capsule formulation.

17. A method for reducing inter-patient and/or intra-patient variability in bioavailability and/or plasma concentrations of the Agent in a patient in need of the Agent comprising orally administering to said patient a pharmaceutical composition according to any one of claims 1 to 16, wherein the Agent is as defined in claim 1.

18. A method for increasing the solubilisation of the Agent in an aqueous medium with a pH value similar to those found in the upper GI tract of a human comprising adding to said aqueous medium a pharmaceutical composition according to any one of claims 1 to 16; wherein the solubilisation of the Agent from the composition is increased compared to the solubilisation of the Agent alone in the same aqueous medium; and wherein the Agent is as defined in claim 1.

19. A method for inhibiting the rate of precipitation of the Agent from an aqueous solution comprising adding to an aqueous medium with a pH similar to the gastric pH in a human, a pharmaceutical composition according to any one of claims 1 to 16; wherein the Agent is as defined in claim 1.
